(19) United States
(12) Patent Application Publication
(10) Pub. No.: US 2015/0080644 A1
(43) Pub. Date: Mar. 19, 2015

(54) IMPLANT AND METHOD OF PLACING IMPLANT INDWELLING

(71) Applicant: TERUMO KABUSHIKI KAISHA, Shibuya-ku (JP)

(72) Inventors: Masakatsu KAWAURA, Sunnyvale, CA (US); Nao YOKOI, Sunnyvale, CA (US)

(73) Assignee: TERUMO KABUSHIKI KAISHA, Tokyo (JP)

(21) Appl. No.: 14/489,889

(22) Filed: Sep. 18, 2014

(30) Foreign Application Priority Data
Sep. 18, 2013 (JP) 2013-193161

(51) Int. Cl. A61F 2/00

(52) U.S. Cl. CPC A61F 2/0045
USPC 600/30

(57) ABSTRACT

An implant and a method of placing an implant indwelling are disclosed. The implant, to be placed indwelling in a living body, is inserted into a medical tube prior to being placed indwelling. The implant can include an implant main body which is flexible, is elongated, and is shorter than a length of the medical tube; a guide portion which, at the time of insertion of the implant main body into the medical tube, is inserted into the medical tube prior to the implant main body and guides the implant main body; and an interlock portion which interlock the implant main body and the guide portion to each other, the interlock being released after the implant main body is inserted into the medical tube.

### CROSS-REFERENCES TO RELATED APPLICATIONS

This application claims priority to Japanese Application No. 2013-193161 filed on Sep. 18, 2013, the entire content of which is incorporated herein by reference.

### TECHNICAL FIELD

The present disclosure relates to an implant and a method of placing an implant indwelling.

### BACKGROUND DISCUSSION

In a patient suffering from urinary incontinence, for example, stress urinary incontinence, urine leakage (involuntary urination) occurs due to an abnormal pressure exerted during a normal exercise or by laughing, coughing, sneezing or the like. This can be attributable, for example, to loosening of a pelvic floor muscle, which is a muscle for supporting a urethra, caused by childbirth or the like.

For treatment of urinary incontinence, surgical therapy can be effective. For example, a tape-shaped implant called a “sling” can be placed indwelling in the body to support the urethra (for example, U.S. Pat. No. 6,911,003). In order to put a sling indwelling in the body, an operator incises a vagina with a surgical knife, dissects a biological tissue (living body tissue) between the urethra and the vagina, and provides communication between the exfoliated biological tissue site and an exterior through an obturator foramen by using a puncture needle or the like. Then, in such a state, the sling is placed indwelling in the body.

If a vaginal wall is once incised, however, there can be a fear that the sling might be exposed to an inside of the vagina via a wound caused by the incision. There can also be a fear that complications might occur which can be caused by an infection via the wound or the like. In addition, since the vaginal wall is incised, an invasiveness of the procedure can be rather great and patient burden can be relatively heavy. In addition, there can be a fear that the urethra or the like can be damaged by a surgical knife in the course of the procedure by the operator, and there can be a fear that the operator himself might damage his fingertip by the surgical knife.

Further, when an implant is placed indwelling in a living body, there may arise a case, depending on a length of the implant, where part of the implant can located near the living body surface, such that the patient may experience pain.

### SUMMARY

An implant and a method of placing an implant indwelling are disclosed by which the burden exerted on a patient when an implant is put indwelling in the living body can be relatively alleviated.

In accordance with an exemplary embodiment, an implant is disclosed to be placed indwelling in a living body, the implant inserted into a medical tube prior to being placed indwelling, the implant including an implant main body which is flexible, elongated, and shorter in length than the medical tube, a guide portion which, at a time of insertion of the implant main body into the medical tube, is inserted into the medical tube prior to the implant main body and guides the implant main body and an interlock portion interlocking the implant main body and the guide portion to each other, the interlocking being released after the implant main body is inserted into the medical tube.

In accordance with an exemplary embodiment, the interlock portion can be loop-like in shape, and the interlocking between the implant main body and the guide portion can be released by cutting an intermediate portion of the interlock portion.

In accordance with an exemplary embodiment, at least part of the medical tube can have a curved portion in a longitudinal direction, and the guide portion is higher in rigidity than the implant main body, is deformable into a curved shape, and is deformed along a curved shape of the curved portion when guiding the implant main body.

In accordance with an exemplary embodiment, the implant main body can have an anchor portion which restricts movement in the longitudinal direction of the implant main body in a state in which the medical tube has been pulled out and the implant main body has been placed indwelling in the living body.

In accordance with an exemplary embodiment, a marker is disclosed, which indicates a position of the implant main body in the living body, the marker being provided on the interlock portion or the guide portion.

In accordance with an exemplary embodiment, wherein the medical tube can be rigid to such an extent as to be able to maintain a lumen when left indwelling in the living body, and the implant main body is more flexible than the medical tube.

In accordance with an exemplary embodiment, a method of placing an implant indwelling in a living body by use of a medical tube in which the implant can be inserted is disclosed, the method including placing the medical tube indwelling in the living body and inserting the implant into the medical tube, and thereafter pulling the medical tube out of the living body while leaving the implant in the living body so that the implant is placed indwelling in the living body.

In accordance with an exemplary embodiment, when an implant is placed indwelling in a living body, the implant can be set indwelling in a deeper position from a body surface, according as an implant main body is shorter in length than a medical tube. Therefore, the burden on the patient can be relatively alleviated.

For example, in the case where the implant main body has anchor portions restricting movement of the implant main body in the longitudinal direction of the implant main body, in the indwelling state in which the implant main body is placed indwelling in a living body, the indwelling state can be reliably maintained.

### BRIEF DESCRIPTION OF THE DRAWINGS

FIG. 1 is a perspective view showing a puncture apparatus to be used at a time of placing indwelling in a living body an implant according to a first exemplary embodiment of the present disclosure.

FIG. 2 is a side view of the puncture apparatus shown in FIG. 1.

FIG. 3 is a plan view showing an operating member possessed by the puncture apparatus shown in FIG. 1.

FIGS. 4A and 4B illustrate a puncture member possessed by the puncture apparatus shown in FIG. 1, wherein FIG. 4A is a perspective view, and FIG. 4B is a sectional view taken along line 4B-4B in FIG. 4A.

FIG. 5 is a sectional view of the puncture member shown in FIG. 4A.

FIGS. 6A to 6C illustrate a state-maintaining mechanism possessed by the puncture member shown in FIG. 4A, wherein FIG. 6A is a top plan view and FIGS. 6B and 6C are sectional views.

FIGS. 7A to 7C are partial magnified views showing the state-maintaining mechanism possessed by the puncture member shown in FIG. 4A, wherein FIGS. 7A and 7B are plan views showing modifications, respectively, and FIG. 7C is a plan view illustrating this embodiment.

FIGS. 8A and 8B illustrate a second anchor possessed by the puncture apparatus shown in FIG. 1, wherein FIG. 8A is a sectional view, and FIG. 8B is a sectional view showing the second anchor in a state of being engaged with the puncture member.

FIGS. 9A and 9B illustrate a first anchor possessed by the puncture apparatus shown in FIG. 1, wherein FIG. 9A is a sectional view, and FIG. 9B is a sectional view showing the first anchor in a state of being engaged with the puncture member.

FIG. 10 is a sectional view showing a guide portion of a frame provided in the puncture apparatus shown in FIG. 1.

FIG. 11 is a sectional view showing the guide portion of the frame provided in the puncture apparatus shown in FIG. 1.

FIG. 12 is a sectional view showing the guide portion of the frame provided in the puncture apparatus shown in FIG. 1.

FIG. 13 is a plan view showing a fixing portion of the frame provided in the puncture apparatus shown in FIG. 1.

FIG. 14 is a side view of an insertion instrument possessed by the puncture apparatus shown in FIG. 1.

FIGS. 15A and 15B illustrate a positional relationship of the puncture member and obturator foramens (pelvis), wherein FIG. 15A is a side view and FIG. 15B is a front view.

FIG. 16 is a partial magnified view of a vaginal insertion member possessed by the insertion instrument shown in FIG. 14.

FIG. 17A is a sectional view showing an example of a shape of a vaginal wall, and FIG. 17B is a sectional view showing a state in which a vaginal insertion portion is inserted in a vagina shown in FIG. 17A.

FIG. 18 is a perspective view showing an implant according to the first embodiment of the present disclosure.

FIGS. 19A and 19B are views for illustrating an operation procedure of the puncture apparatus shown in FIG. 1.

FIGS. 20A and 20B are views for illustrating the operation procedure of the puncture apparatus shown in FIG. 1.

FIG. 21 is a side view showing a relationship between the puncture apparatus and the pelvis at a time of a state shown in FIG. 20A.

FIGS. 22A and 22B are views for illustrating the operation procedure of the puncture apparatus shown in FIG. 1.

FIG. 23 is a side view showing a relationship between the puncture apparatus and the pelvis at a time of a state shown in FIG. 22A.

FIG. 24 is a sectional view showing a posture of the puncture member relative to a urethra at a time of a state shown in FIG. 22B.

FIGS. 25A and 25B are views for illustrating a procedure of placing the implant shown in FIG. 18 indwelling.

FIGS. 26A and 26B are views for illustrating the procedure of placing the implant shown in FIG. 18 indwelling.

FIGS. 27A and 27B are views for illustrating the procedure of placing the implant shown in FIG. 18 indwelling.

FIGS. 28A and 28B are views for illustrating the procedure of placing the implant shown in FIG. 18 indwelling.

FIGS. 29A and 29B are views for illustrating the procedure of placing the implant shown in FIG. 18 indwelling.

FIG. 30 is a perspective view showing an implant according to a second exemplary embodiment of the present disclosure.

FIGS. 31A and 31B are views for illustrating a procedure of placing the implant shown in FIG. 30 indwelling.

FIG. 32 is a perspective view showing an implant according to a third exemplary embodiment of the present disclosure.

FIGS. 33A and 33B are views for illustrating a procedure of placing the implant shown in FIG. 32 indwelling.

FIG. 34 is a plan view showing an implant according to a fourth exemplary embodiment of the present disclosure.

FIG. 35 is a plan view showing an implant according to a fifth exemplary embodiment of the present disclosure.

FIG. 36 is a plan view showing an implant according to a sixth exemplary embodiment of the present disclosure.

FIGS. 37A and 37B are views for illustrating a procedure of placing indwelling an implant according to a seventh exemplary embodiment of the present disclosure.

FIGS. 38A and 38B are views for illustrating a procedure of placing indwelling an implant according to an eighth exemplary embodiment of the present disclosure.

FIGS. 39A and 39B are views for illustrating a procedure of placing indwelling an implant according to a ninth exemplary embodiment of the present disclosure.

FIGS. 40A and 40B are views for illustrating a procedure of placing indwelling an implant according to a tenth exemplary embodiment of the present disclosure.

### DETAILED DESCRIPTION

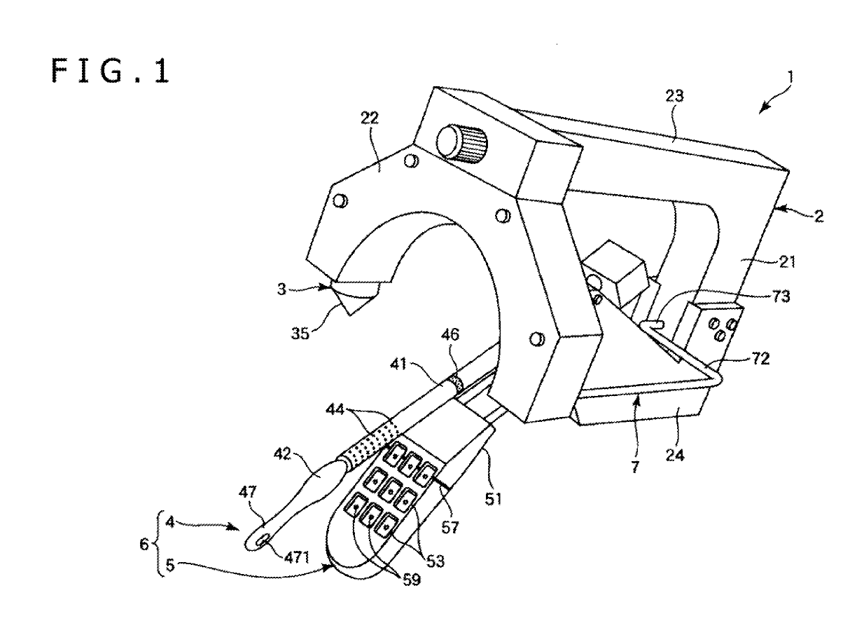

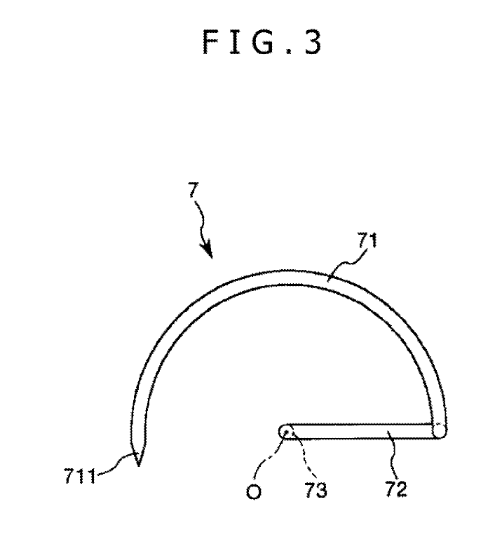

FIGS. 1-29 are views showing a puncture apparatus to be used at a time of placing indwelling in a living body an implant according to a first exemplary embodiment of the present disclosure.

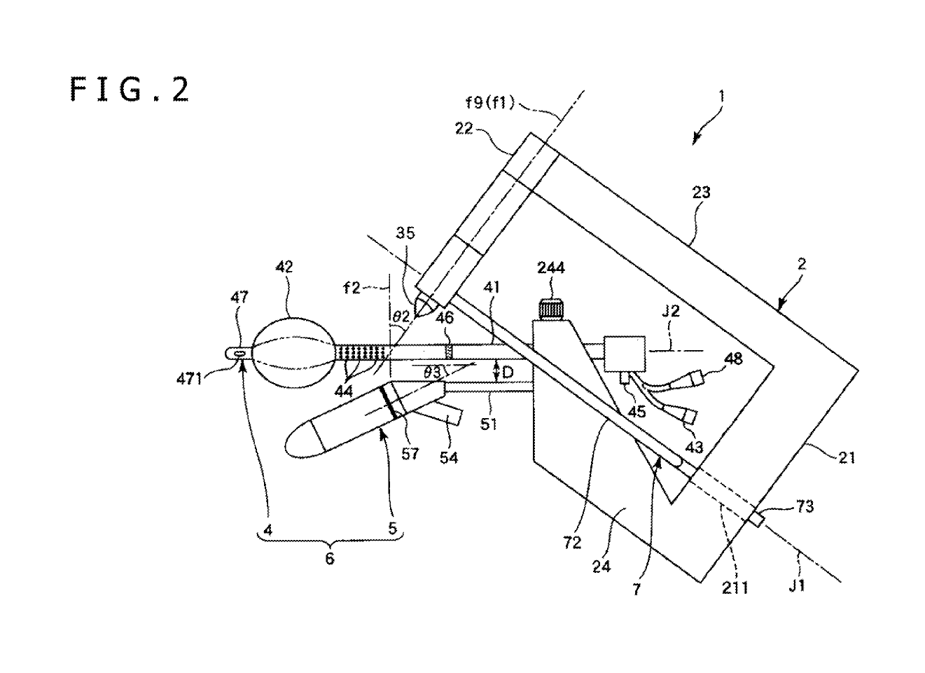

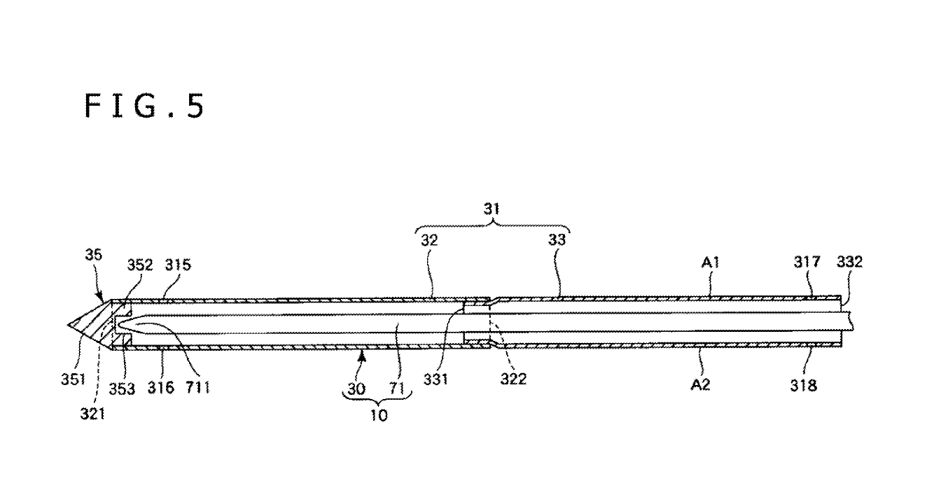

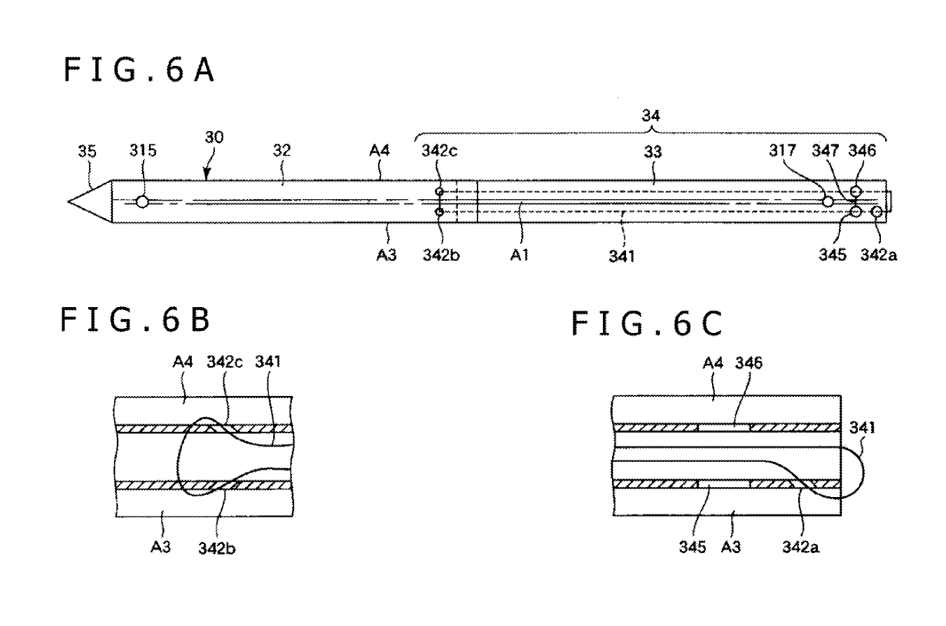

In the following, for convenience of description, the left side in FIG. 2 will be referred to as “distal (side),” the right side as “proximal (side),” the upper side as “upper (side),” and the lower side as “lower (side).” In addition, FIG. 2 shows the puncture apparatus in a state of being yet to be used, which state will hereinafter be referred to also as “initial state,” for convenience of description. Further, a state wherein the puncture apparatus (insertion instrument) shown in FIG. 2 is mounted on a patient will be referred to also as “mounted state.” In addition, in FIGS. 5 to 6C, for convenience of description, the puncture member actually extending in an arcuate shape is depicted in a rectilinearly stretched state.

In accordance with an exemplary embodiment, a puncture apparatus to be used at a time of placing an implant of the present disclosure indwelling in a living body is disclosed.

In accordance with an exemplary embodiment, a puncture apparatus 1 as shown in FIGS. 1 and 2 is an apparatus to be used for treatment of female urinary incontinence, for example, to be used in a process in which a biological tissue-supporting implant for treatment of urinary incontinence is embedded (implanted) into a living body.

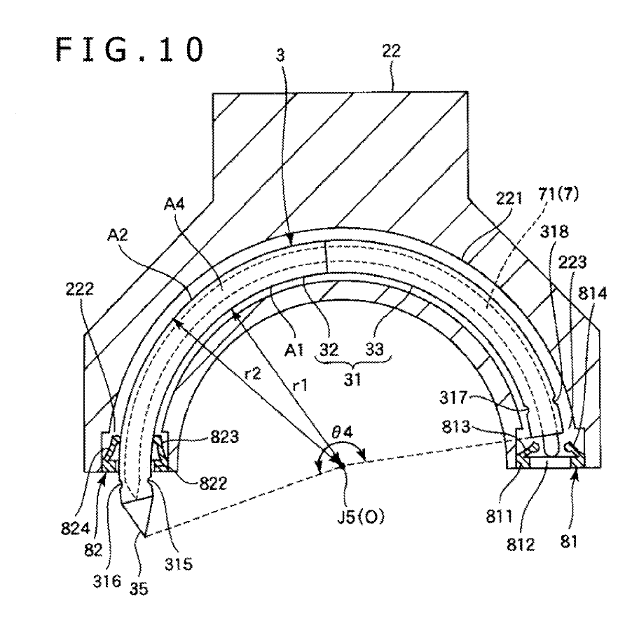

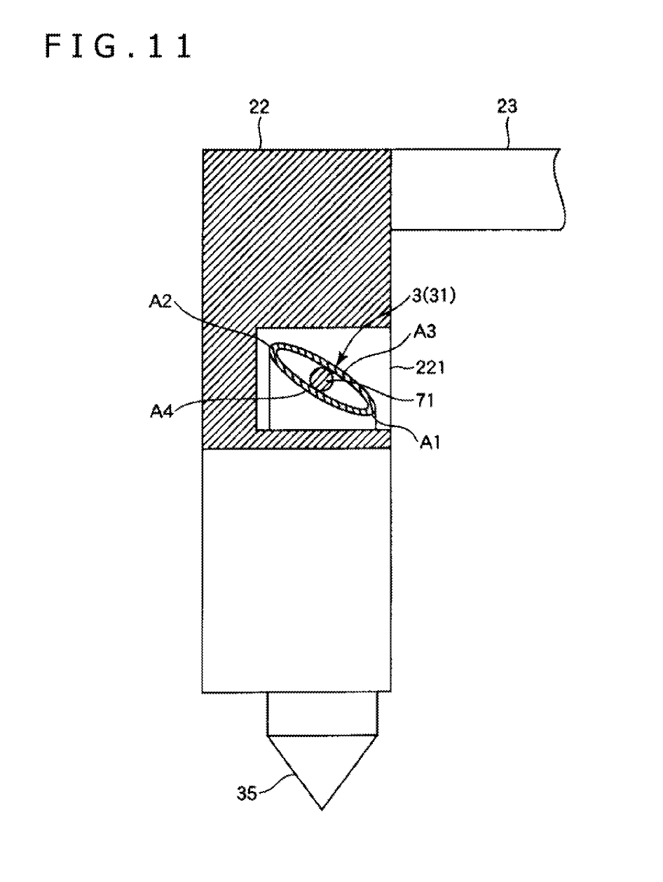

The puncture apparatus 1 can include a frame (support unit) 2, a puncture member 3, a urethral insertion member 4, a vaginal insertion member 5, an operating member 7 and anchors 81 and 82. The puncture member 3, the urethral insertion member 4, the vaginal insertion member 5, the operating member 7, and the anchors 81 and 82 can be supported on the frame 2 (see FIG. 10). In the puncture apparatus 1, the urethral insertion member 4 and the vaginal insertion member 5 constitute an insertion instrument 6.

The operating member 7 is a member operating the puncture member 3. Such an operating member 7, as shown in FIGS. 1 to 3, can include an insertion portion 71, a shaft portion 73, and an interlock portion 72 interlocking the insertion portion 71 and the shaft portion 73 to each other. The insertion portion 71, the interlock portion 72, and the shaft portion 73 may be formed integrally, or, alternatively, at least one of the portions may be formed as a separate body in relation to the other portions.

The insertion portion 71 is a portion to be inserted in the puncture member 3, and can function as a stylet that supports the puncture member 3 from the inside. With the insertion portion 71 inserted in the puncture member 3, the puncture member 3 is connected to the operating member 7, whereby it is enabled to operate the puncture member 3 by the operating member 7. Such an insertion portion 71 is in an arcuate shape corresponding to the shape of the puncture member 3. A center angle of the insertion portion 71 is set in conformity with a center angle of the puncture member 3. In accordance with an exemplary embodiment, a distal portion 711 of the insertion portion 71 can be tapered off. The presence of the tapered distal portion 711 can enable relatively smooth fitting of the puncture member 3 over the insertion portion 71 (smooth insertion of the insertion portion 71 into the puncture member 3).

In accordance with an exemplary embodiment, the insertion portion 71 can be circular in cross-sectional shape. Alternatively, the insertion portion 71 may be flat-shaped in cross section. The flat shape is not limited. Examples of the flat shape applicable here can include not only ellipses but also rounded-cornered rhombuses, rounded-cornered rectangles (flat shapes), and spindle-like shapes enlarged (enlarged in diameter) at a central portion as compared with both end portions of the insertion portion being flat-shaped in cross section.

The shaft portion 73 can extend along an axis J1 which intersects a center O of the insertion portion 71 and which is orthogonal to a plane f1 that contains the insertion portion 71.

The interlock portion 72 interlocks a proximal portion of the insertion portion 71 and a distal portion of the shaft portion 73 to each other. In addition, the interlock portion 72 can be substantially L-shaped, with a substantially rectangular bend at an intermediate portion of the interlock portion 72. The interlock portion 72 can function as a grasping portion to be grasped by an operator at a time of operating the operating member 7.

In accordance with an exemplary embodiment, such an operating member 7 can be configured to be higher than the puncture member 3 (sheath main body 31) in rigidity. The material constituting the operating member 7 is not limited. Examples of the material applicable here can include various metallic materials such as stainless steels, aluminum, aluminum alloys, titanium, and titanium alloys.

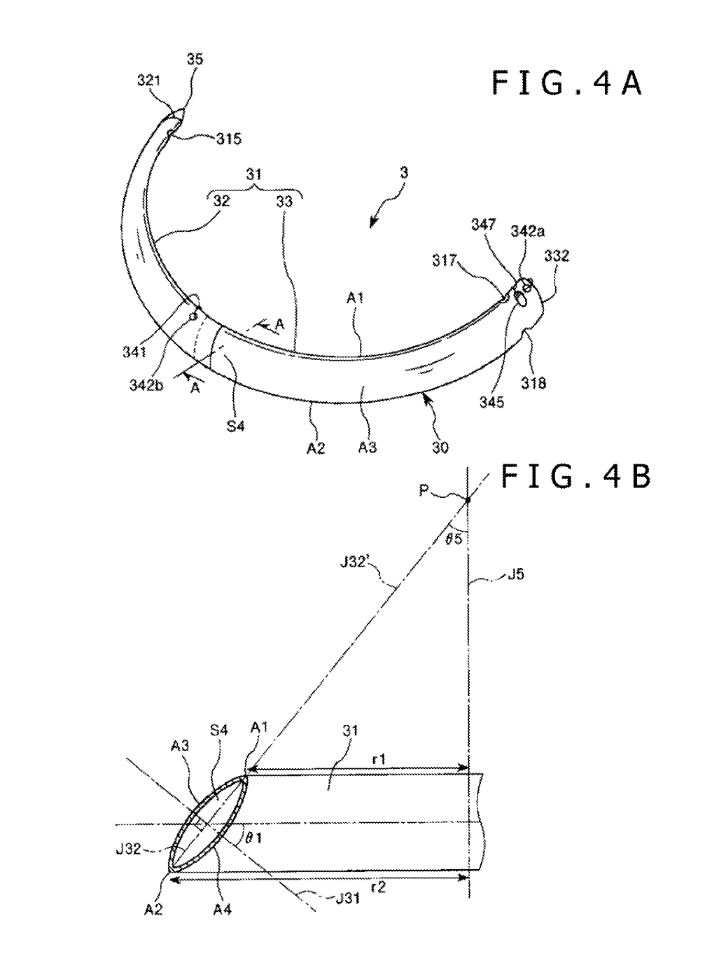

The puncture member 3 is a member puncturing a living body. Such a puncture member 3, as shown in FIG. 4A, can include an elongate sheath (medical tube) 30, and a needle body 35 provided at a distal end of the sheath 30. In addition, the sheath 30 can include a tube-shaped sheath main body 31, and a state-maintaining mechanism 34, and is inserted into a living body prior to a time when the implant 9 according to the present disclosure is put indwelling in the living body.

The sheath main body 31 can be configured by use of an elongate tube, which is open at a distal end and a proximal end of the elongate tube. Such a sheath main body 31 can have an internal space in which an implant main body 91 can be inserted. In addition, the sheath main body 31 can be in an arcuate curved shape, and can have a flat cross-sectional shape as shown in FIG. 4B. For example, the cross-sectional shape of the sheath main body 31 at a central portion S4 of in the longitudinal direction of the sheath main body 31 is a flat shape, which can include a minor axis J31 and a major axis J32. As will be described later, the implant main body 91 (implant 9) is inserted in the sheath main body 31 (this state will hereinafter be referred to as “inserted state”). With the sheath main body 31 set in the flat shape, therefore, a posture of the implant main body 91 inside the sheath main body 31 can be controlled.

In accordance with an exemplary embodiment, a width (a length in the direction of the major axis J32) of the internal space of the sheath main body 31 can be designed to be approximately equal to a width of the implant main body 91. This help ensure that even when the implant main body 91 is moved, a frictional resistance between the implant main body 91 and the internal space of the sheath main body 31 is lowered, and no unnecessary force is applied to the implant main body 91. Accordingly, the implant main body 91 can be disposed in a sufficiently expanded (spread) state in the inside of the sheath main body 31. The width (the length in the direction of the major axis J32) of the internal space of the sheath main body 31 may be shorter than the width of the implant main body 91, which helps ensure that the width of the sheath main body 31 is set smaller, so that a less invasive puncture member 3 can be realized.

The flat shape of the sheath main body 31 is not limited. Examples of the flat shape applicable here can include ellipses, sectionally convexed lens-like shapes, rounded-cornered rhombuses, rounded-cornered rectangles (flat shapes), and spindle-like shapes enlarged (enlarged in diameter) at a central portion as compared with both end portions

Hereinafter, for convenience of description, as shown in FIG. 4B, an end portion located on an inner side (one end portion) in the direction of the major axis J32 will be referred to also as “inner circumferential portion A1,” an end portion located on an outer side (other end portion) will be referred to also as “outer circumferential portion A2,” a surface oriented upward will be referred to also as “front surface A3,” and a surface oriented downward will be referred to also as “back surface A4.”

As shown in FIGS. 2 and 4B, when a plane containing both a center of the arc of the central portion S4 and a center of the cross-sectional shape relative to the longitudinal direction of the sheath main body 31 (a plane containing a center axis of the sheath main body 31) is referred to as plane f9 and an angle formed between the plane f9 and the minor axis J31 at the central portion S4 is referred to as inclination angle θ1, the inclination angle θ1 is preferably an acute angle. With the inclination angle θ1 set to be an acute angle, the implant 9 (described later) can be disposed substantially in parallel to the urethra, whereby the urethra can be supported relatively effectively.

The inclination angle θ1 is not limited, insofar as it is an acute angle. The inclination angle θ1 can be, for example, about 20 to 60 degrees, more preferably about 30 to 45 degrees, and further preferably about 35 to 40 degrees, which helps ensure that the above-mentioned effect is further enhanced.

The inclination angle θ1 is configured to satisfy an above-mentioned numerical range throughout a whole region in the extending direction of the sheath main body 31. However, the above-mentioned effect can be displayed if only the inclination angle θ1 satisfies the above-mentioned numerical range at least in the central portion S4 in the extending direction of the sheath main body 31. The central portion S4″ refers to a region that includes a part located between the urethra and the vagina, at least, in a state in which the puncture member 3 is puncturing a living body (a state wherein the sheath main body 31 is disposed in a living body). It can also be said that in this exemplary embodiment, a central portion (the middle and the vicinity on both sides of the middle) between the anchors 81 and 82, in a state wherein the anchors 81 and 82 are engaged with the puncture member 3 as described later, is the central portion S4.

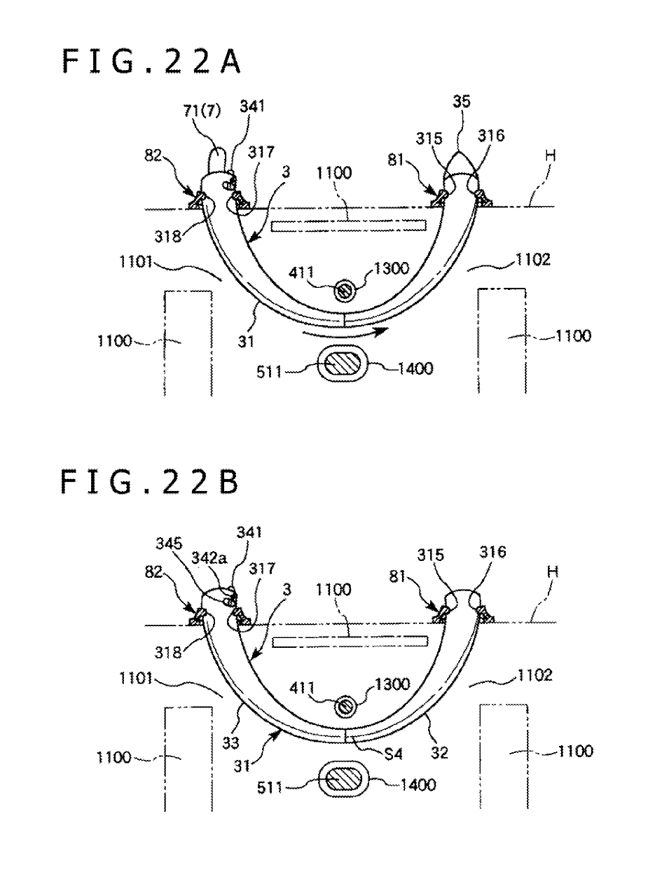

In accordance with an exemplary embodiment, the sheath main body 31 may be provided at both end portions of the sheath main body 31 with markers located at parts which are equally spaced from the central portion S4 and which are protruding to the outside of a living body in a state in which the sheath main body 31 is disposed in the living body (a state as shown in FIGS. 22A and 22B), which helps ensure that it is possible, by comparing the positions of both the markers with each other, to check the position of the central portion S4 in the inside of the living body.

The configuration of the sheath main body 31 can also be expressed as follows. As shown in FIG. 4B, the sheath main body 31 is so formed that the major axis J32 can be inclined against a center axis J5 of the arc and that the center axis J5 of the arc and an extension line J32′ of the major axis J32 have an intersection P. In accordance with an exemplary embodiment, an angle θ5 formed between the center axis J5 and the extension line J32′ is equal to the inclination angle θ1. As shown in FIG. 10, the sheath main body 31 has the inner circumferential portion A1 located at an inner circumferential edge (in plan view as viewed from a direction of the center axis J5 of the sheath main body 31) and having a minimum radius of curvature, r1, and also has the outer circumferential portion A2 located at an outer circumferential edge (in the plan view) and having a maximum radius of curvature, r2, and that the inner circumferential portion A1 and the outer circumferential portion A2 are located to be spaced (deviated) from each other along the direction of the center axis J5.

The sheath main body 31 can be composed of two split pieces to that it can be split at an intermediate portion of the sheath main body 31. For example, the sheath main body 31 can be divided into a distal split piece 32 and a proximal split piece 33. The distal split piece 32 and the proximal split piece 33 can be approximately equal in length, and their boundary can be located at the central portion S4.

As shown in FIG. 5, the distal split piece 32 is tubular in shape, and has a distal end opening 321 and a proximal end opening 322. For example, the proximal split piece 33 can be tubular in shape, and can have a distal end opening 331 and a proximal end opening 332. A distal portion of the proximal split piece 33 can be inserted into a proximal portion of the distal split piece 32, whereby the distal split piece 32 and the proximal split piece 33 are connected to each other. With the proximal split piece 33 thus inserted into the distal split piece 32, a step which can be generated at a boundary between the split pieces 32 and 33 is insusceptible to be caught on a biological tissue, so that puncturing of a living body by the puncture member 3 can be performed smoothly. In accordance with an exemplary embodiment, the distal split piece 32 may be inserted into the proximal split piece 33 so as to connect the split pieces 32 and 33 to each other.

The connected state of the split pieces 32 and 33 thus connected can be maintained by the state-maintaining mechanism 34. As shown in FIG. 6A, the state-maintaining mechanism 34 can include holes 342a, 342b, and 342c, an endless string (interlock member) 341 passed through the holes 342a, 342b, and 342c, exposure holes (through-holes) 345 and 346 for exposing the string 341 therethrough, and a slit 347 joining the exposure holes 345 and 346 to each other.

The hole 342a can be provided in a proximal portion of the proximal split piece 33, for example, at the front surface A3 near the inner circumferential portion A1. In accordance with an exemplary embodiment, the holes 342b and 342c can be oppositely provided in a proximal portion of the distal split piece 32, for example, at the front surface A3 and the back surface A4 near the inner circumferential portion A1.

The string 341 can be disposed inside the sheath main body 31, and can be exposed to the outside of the sheath main body 31, between the hole 342b and the hole 342c, and between the hole 342a and the proximal end opening 332. With the string 341 laid around in this manner, the connected state of the split pieces 32 and 33 can be maintained securely. In addition, the exposure of the string 341 to the outside of the sheath main body 31 can be suppressed, so that the string 341 is less liable to be caught on a biological tissue. In accordance with an exemplary embodiment, while making it possible to cut the string 341 as will be described later, an overall length of the string 341 can be made as short as possible. Therefore, at the time of inserting and passing an implant main body 91 into and through the sheath main body 31, the string 341 is less liable to be caught on the implant main body 91. In addition, since the holes 342a, 342b, and 342c can be disposed near the inner circumferential portion A1 as above-mentioned, the string 341 can also be disposed near the inner circumferential portion A1, which can help ensure that at the time of inserting the implant main body 91 into the sheath main body 31, the string 341 is less liable to be caught on the implant main body 91.

The string 341 can be obtained, for example, in the following manner. A string having ends can be prepared where one end of the string is inserted through the proximal end opening 332 into the inside of the sheath main body 31, is drawn out via the hole 342b to the outside of the sheath main body 31, is inserted through the hole 342c into the inside of the sheath main body 31, is drawn out via the hole 342a to the outside of the sheath main body 31, and is finally tied with the other end of the string near the proximal end opening 332. In accordance with an exemplary embodiment, the position of the knot is not limited.

As shown in FIG. 6C, an axis of the hole 342a can be so inclined that an outside opening of the hole 342a is located on the proximal end of an inside opening of the hole 342a. As shown in FIG. 6B, an axis of each of the holes 342b and 342c can be so inclined that an outside opening of the hole is located on the distal end of an inside opening of the hole, which helps enable the holes 342a, 342b, and 342c to extend along the course of the string 341, so that the string 341 is less liable to be caught on the holes 342a, 342b, and 342c.

The exposure holes 345 and 346 can be oppositely provided in the front surface A3 and in the back surface A4, at a proximal portion of the proximal split piece 33. The part where the exposure holes 345 and 346 are provided protrudes from a body surface, in the condition where the sheath main body 31 is disposed in a living body. Further, the exposure holes 345 and 346 are located on the course of the string 341. Therefore, the string 341 can be exposed via the exposure holes 345 and 346 to the outside of the sheath main body 31. In addition, the exposure holes 345 and 346 are joined to each other by the slit 347, which can be provided in the inner circumferential portion A1 along the circumferential direction of the sheath main body 31.

In the state-maintaining mechanism 34 as above, cutting the string 341 can result in a state wherein the distal split piece 32 and the proximal split piece 33 can be separated from each other. This configuration can help ensure that the distal split piece 32 and the proximal split piece 33 can be put into a separable state by a relatively simple operation. In addition, since the cutting of the string 341 can be visually checked, it can be confirmed relatively easily that the distal split piece 32 and the proximal split piece 33 have been put into the separable state.

The exposure holes 345 and 346 and the slit 347 thus provided, as in this exemplary embodiment, can enable relatively easy cutting of the string 341. In one example, a pair of scissors having a pair of cutting blades (a first blade and a second blade) is prepared. The first blade is inserted and passed in the exposure holes 345 and 346, and the string 341 is located between the pair of blades. Then, the scissors is put into a closing operation, whereon at least one of the first and second blades passes the slit 347, and the first and second blades overlap each other. During this process, the string 341 is cut. In this way, the exposure holes 345 and 346 and the slit 347 provided as above-mentioned enable relatively easy cutting of the string 341.

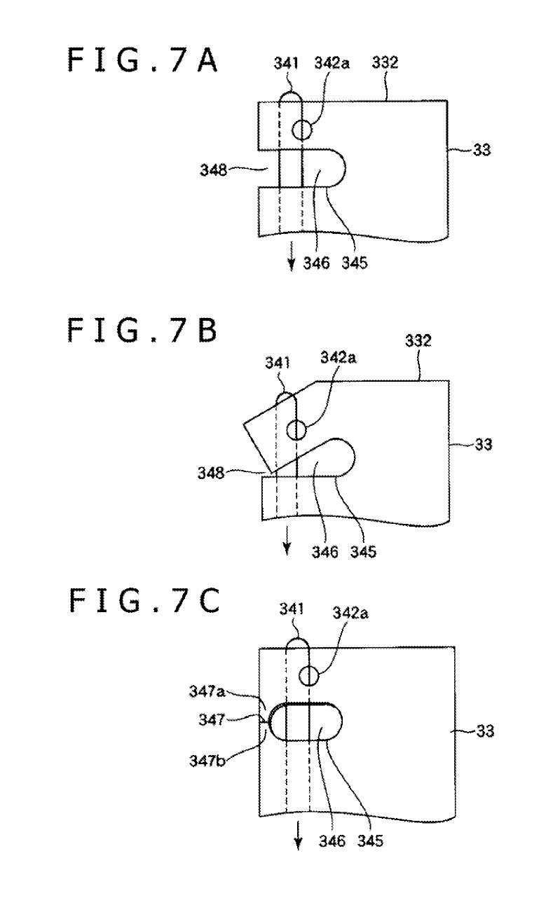

Thus, in this exemplary embodiment, the slit 347 is provided, and the slit 347 is used as a passing route for the blade, which can help ensure that deformation of the sheath main body 31 due to tension on the string 341 is prevented. For example, as shown in FIG. 7A, the passing route for the blade may be configured by use of a hole 348 in place of the slit 347. In this case, however, the hole 348 may, depending on the hardness of the sheath main body 31 or the like factor, be collapsed through buckling under the tension on the string 341, and the sheath main body 31 may be deformed thereby, as shown in FIG. 7B. In the case of the slit 347, for example, those parts 347a and 347b on opposite sides of the slit 347 abut against each other, so that the above-mentioned deformation would not occur, and deformation of the sheath main body 31 can be prevented, as shown in FIG. 7C.

In addition, in as shown in FIG. 5, the sheath main body 31 is provided at its distal portion with a pair of engaging holes 315 and 316 for engagement with the anchor 81. For example, the sheath main body 31 can be provided at its proximal portion with a pair of engaging holes 317 and 318 for engagement with the anchor 82. Of these four engaging holes, the engaging holes 315 and 317 can be provided in the inner circumferential portion A1, whereas the engaging holes 316 and 318 can be provided in the outer circumferential portion A2.

As disclosed, the sheath main body 31 can be flat in shape, and is less liable to be collapsed in the major axis direction of the sheath main body 31. Therefore, the spacing between the inner circumferential portion A1 and the outer circumferential portion A2 can be insusceptible to variation. In addition, the inner circumferential portion A1 and the outer circumferential portion A2 can be greater in curvature and can be less susceptible to deformation, as compared with the front surface A3 and the back surface A4. Therefore, with the engaging holes 315 and 317 provided in the inner circumferential portion A1 and with the engaging holes 316 and 318 provided in the outer circumferential portion A2, it can help ensure that the engagement between the anchors 81 and 82 and the sheath main body 31 is less liable to be released (canceled).

In accordance with an exemplary embodiment, the spacing between the engaging holes 315 and 316 and the central portion S4 and the spacing between the engaging holes 317 and 318 and the central portion S4 are approximately equal to each other. This help ensure that the anchors 81 and 82 play the role of markers, which can enable relatively easy grasping of the position of the central portion S4 of the sheath main body 31 in the inside of a living body.

The sheath main body 31 as disclosed above can be provided with the needle body 35 at the distal end of the sheath main body 31. As shown in FIG. 5, the needle body 35 has a needle tip 351, which is tapered off, and a proximal portion 352 provided on the proximal end of the needle tip 351. The proximal portion 352 is inserted in the sheath main body 31, whereby the needle body 35 is held inside the sheath main body 31 in a freely detachable manner. The proximal portion 352 can be fitted in the sheath main body 31 with such a force that unintended detachment of the needle body 35 from the sheath main body 31 can be relatively prevented. The needle body 35 may be configured integrally with the sheath main body 31.

In addition, the proximal portion 352 can be provided with an engaging portion 353 for engagement with a distal portion 711 of the insertion portion 71. The engaging portion 353 can be composed essentially of a recess. In an inserted state wherein the puncture member 3 is inserted in the insertion portion 71, the distal portion 711 is located inside the engaging portion 353. The engaging portion 353 thus provided helps ensure that displacement of the needle body 35 relative to the insertion portion 71 is relatively suppressed, and puncturing of a living body by the puncture member 3 can be carried out smoothly.

For example, where at least the distal portion 711, of the insertion portion 71, is flat-shaped in cross section, it can be preferable that the cross-sectional shape of the engaging portion 353 is set in conformity with the cross-sectional shape of the distal portion 711, for example, it can be preferable that the engaging portion 353 is also flat-shaped in cross section. This helps ensure that when the engaging portion 353 and the distal portion 711 are in engagement with each other, the flat shape of the engaging portion 353 and the flat shape of the distal portion 711 overlap each other. This overlap helps ensure that the sheath 30 is restrained from rotating relative to the insertion portion 71, about the axis of the sheath 30.

In the above, the puncture member 3 has been described. A center angle θ4 of the puncture member 3 is not limited, and can be set, as necessary, according to various conditions. For example, as will be described later, the center angle θ4 can be set that the needle body 35 can enter a body via a patient's inguinal region on one side, pass between the urethra and the vagina and protrude to the outside of the body via an inguinal region on the other side. For example, the center angle θ4 can be in the range of about 150 to 270 degrees, more preferably 170 to 250 degrees, and further preferably 190 to 230 degrees.

For example, the material constituting each of the sheath main body 31 and the needle body 35 can be a material being rigid to such an extent that it can maintain the shape and an internal space (lumen) of the sheath main body 31 or the needle body 35 in the state of being inserted in a body. Examples of such rigid material applicable here include various resin materials such as polyethylene, polyimides, polyamides, polyester elastomers, polypropylene, etc. and various metallic materials such as stainless steels, aluminum, aluminum alloys, titanium, and titanium alloys. For example, instead of adopting rigid materials constituting the sheath main body 31 and the needle body 35, other materials than rigid materials may also be adopted, in which case a wall of the sheath main body 31 or the needle body 35 is reinforced with a reinforcement member, whereby the preferable properties as disclosed above can be attained. For example, a high-strength braiding may be embedded in the wall of the sheath main body 31 or the needle body 35, whereby the shape and the internal space of the sheath main body 31 or the needle body 35, in the state of being inserted in a body, can be maintained. In accordance with an exemplary embodiment, the reinforcement member can be a spiral body, which may be embedded in the wall of the sheath main body 31, whereby the sheath main body 31 can have flexibility while retaining the internal space to such an extent that an inserted body can be slid therein.

In accordance with an exemplary embodiment, the sheath main body 31 can be light-transmitting so that the inside of the sheath main body 31 is externally visible, which can make it possible to check, for example, whether or not the distal portion 711 of the insertion portion 71 inserted in the sheath main body 31 is in engagement with the engaging portion 353, or whether or not the string 341 has been cut.

The puncture member 3 (sheath main body 31) as disclosed above and the insertion portion 71 to be inserted in the sheath main body 31 constitute a medical tube assembly 10, and the puncture apparatus 1. The use of the puncture apparatus 1 is started in the state in which they have been assembled into the medical tube assembly 10.

In accordance with an exemplary embodiment, the number and layout of the holes (342a, 342b, 342c) through which to pass the string 341 are not limited, insofar as the connected state of the distal split piece 32 and the proximal split piece 33 can be maintained by the string 341. In addition, the string 341 may not necessarily be in an endless form, but may have ends, namely, one end and the other end. For example, a string having ends may be prepared, its one end may be passed through the hole 342a and the proximal end opening 332 and be looped, and the other end may be passed through the holes 342b and 342c and be looped. Furthermore, the string 341 can include braiding, bands, and the like that can be used in the same manner as the string 341.

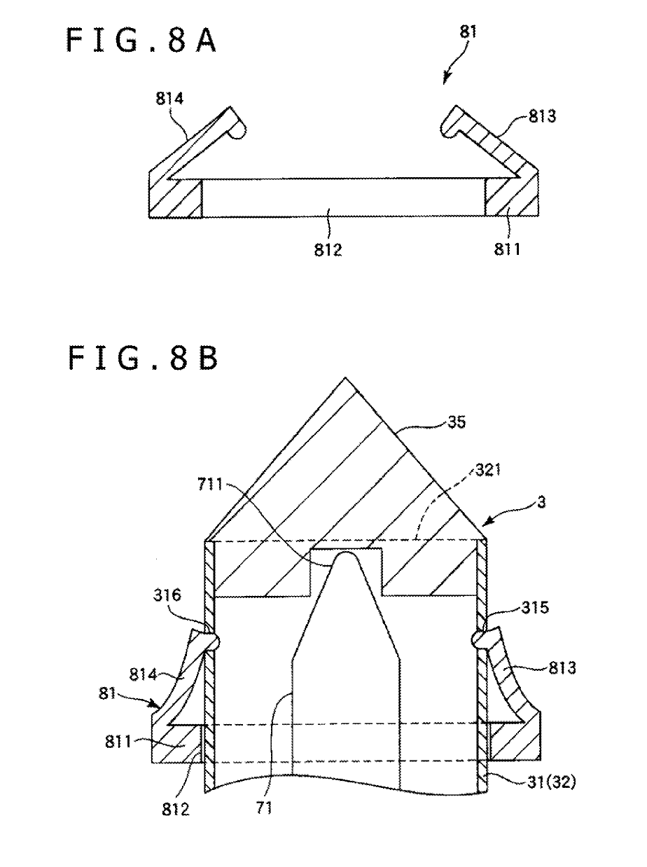

As shown in FIG. 8A, the anchor (second anchor) 81 can include a base portion 811 having a passing hole 812 through which to pass the sheath main body 31, and a pair of claw portions 813 and 814 which project from the base portion 811 and are engaged with the pair of engaging holes 315 and 316. The cross-sectional shape of the passing hole 812 can correspond to the cross-sectional shape of the sheath main body 31. Therefore, in a state in which the puncture member 3 is inserted and passed through the passing hole 812, rotation of the anchor 81 relative to the puncture member 3 is restricted, so that the positional relationship of these components is appropriately maintained. When the puncture member 3 is inserted into the passing hole 812 and the puncture member 3 is advanced relative to the anchor 81 by pushing, the claw portions 813 and 814 are engaged with the engaging holes 315 and 316, as shown in FIG. 8B. This causes the anchor 81 to be engaged with the distal split piece 32. In the engaged state, the base portion 811 is located on the proximal end of the claw portions 813 and 814. As disclosed above, in the state in which the puncture member 3 is passed through the passing hole 812, the rotation of the anchor 81 relative to the puncture member 3 can be restricted. Therefore, engagement between the claw portions 813 and 814 and the engaging holes 315 and 316 can be relatively assured.

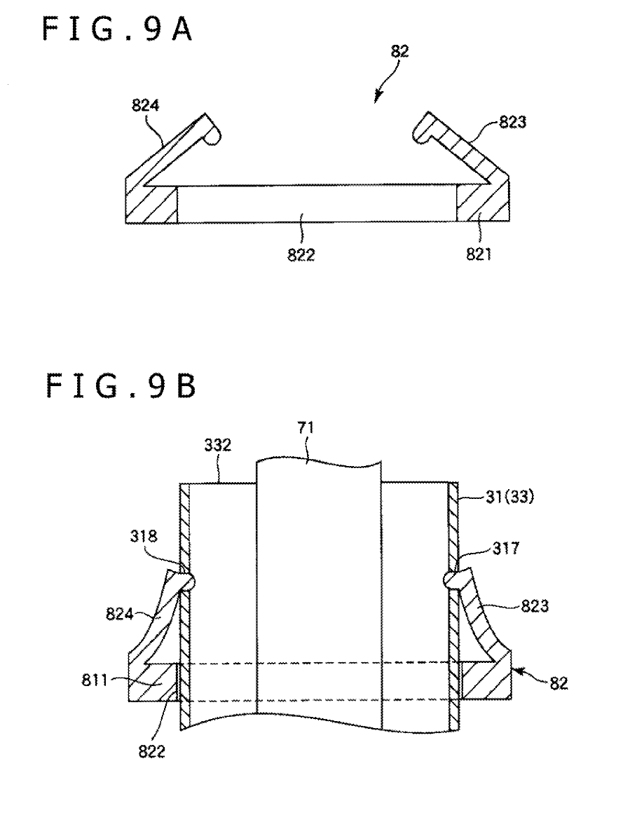

As shown in FIG. 9A, the anchor (first anchor) 82 can include a base portion 821 having a passing hole 822 through which to pass the sheath main body 31, and a pair of claw portions 823 and 824 which project from the base portion 821 and can be engaged with the pair of engaging holes 317 and 318. The cross-sectional shape of the passing hole 822 corresponds to the cross-sectional shape of the sheath main body 31. Therefore, in a state in which the puncture member 3 is inserted and passed through the passing hole 822, rotation of the anchor 82 relative to the puncture member 3 can be restricted, and the positional relationship between these components can be appropriately maintained. When the puncture member 3 is inserted into the passing hole 812 and the puncture member 3 is advanced relative to the anchor 82 by pushing, the claw portions 823 and 824 are engaged with the engaging holes 317 and 318, as shown in FIG. 9B, which causes the anchor 82 to be engaged with the proximal split piece 33. In the engaged state, the base portion 821 is located on the distal end of the claw portions 823 and 824. As disclosed above, in the state in which the puncture member 3 is passed through the passing hole 822, the rotation of the anchor 82 relative to the puncture member 3 can be restricted. Therefore, engagement between the claw portions 823 and 824 and the engaging holes 317 and 318 can be reliably developed.

The materials constituting the anchors 81 and 82 are not limited. For example, various resin materials can be used.

The frame 2 turnably holds the operating member 7 on which the puncture member 3 is mounted. In addition, the frame 2 detachably fixes the insertion instrument 6 and the anchors 81 and 82. The frame 2 has a function of determining a puncture route for the needle body 35 at the time of puncturing of a biological tissue by the puncture member 3. For example, the frame 2 can determine a positional relationship between the puncture member 3, the urethral insertion member 4, and the vaginal insertion member 5 in such a manner that when a biological tissue is punctured by the puncture member 3, the needle body 35 can pass between the urethral insertion member 4 and the vaginal insertion member 5 without colliding against any of the insertion members 4 and 5.

As shown in FIGS. 1 and 2, the frame 2 can include a bearing portion 21 bearing the shaft portion 73 of the operating member 7, a guide portion (holding portion) 22 guiding the puncture member 3 and detachably holding the first and second anchors 81 and 82, an interlock portion 23 interlocking the bearing portion 21 and the guide portion 22 to each other, and a fixing portion 24 to which the insertion instrument 6 is fixed.

The bearing portion 21 can be located on the proximal end in the puncture apparatus 1, and extends in a direction substantially orthogonal to the axis J1. The bearing portion 21 can be formed with a through-hole 211 in a position on the axis J1, and the shaft portion 73 can be turnably inserted in the through-hole 211, which helps ensure that the operating member 7 is supported on the frame 2 in a state of being turnable about the axis J1.

The guide portion 22 can be located on the distal end in the puncture apparatus 1, and is disposed opposite to the bearing portion 21. As shown in FIG. 10, the guide portion 22 is formed with a roughly C-shaped guide groove 221 accommodating the puncture member 3 and guiding the puncture member 3. In addition, as shown in FIG. 11, when disposed inside the guide groove 221, the puncture member 3 has the back surface A4 located on the distal end and has the front surface A3 located on the proximal end.

The guide portion 22 detachably holds the anchors 81 and 82. The anchor 82 is held opposite to the distal end opening 222 so that the passing hole 822 is continuous with the guide groove 221. The anchor 81 is held opposite to the proximal end opening 223 so that the passing hole 812 is continuous with the guide groove 221.

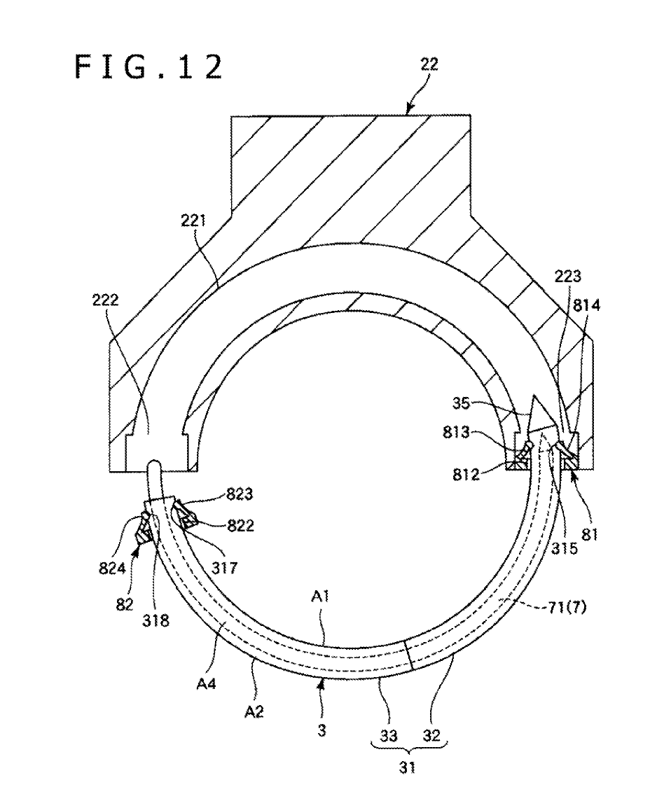

In an initial state, the sheath main body 31 is passed through the passing hole 822 in the anchor 82, and the needle body 35 is protruding from the guide portion 22. When a rotating operation is applied to the operating member 7, the puncture member 3 gradually protrudes from the guide portion 22, and, the needle body 35 enters into the guide portion 22 via the proximal end opening 223, as shown in FIG. 12. In this process, on the distal end of the puncture member 3, the puncture member 3 passes through the passing hole 812 of the anchor 81, and the claw portions 813 and 814 are engaged with the engaging holes 315 and 316. On the proximal end of the puncture member 3, the claw portions 823 and 824 are engaged with the engaging holes 317 and 318, which results in the anchors 81 and 82 engaging with the puncture member 3.

The interlock portion 23 can interlock the shaft portion 21 and the guide portion 22 to each other. In addition, the interlock portion 23 can be in the shape of a bar extending substantially in parallel to the axis J1. The interlock portion 23 can also function as a grasping portion, allowing an operator to use the puncture apparatus 1 while grasping the interlock portion 23.

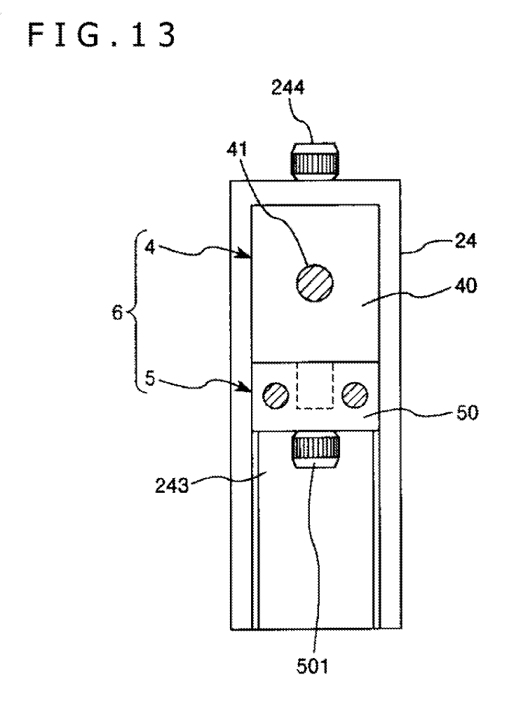

The fixing portion 24 is disposed opposite to the interlock portion 23, with the axis J1 interposed therebetween. As shown in FIG. 13, the fixing portion 24 can have a recess 243 in which to fit a support portion 60 (described later) of the insertion instrument 6, and a male screw 244. With the support portion 60 fitted in the recess 243 and with the male screw 244 fastened into a female screw (not shown) in the support portion 60, the insertion instrument 6 can be fixed to the fixing portion 24.

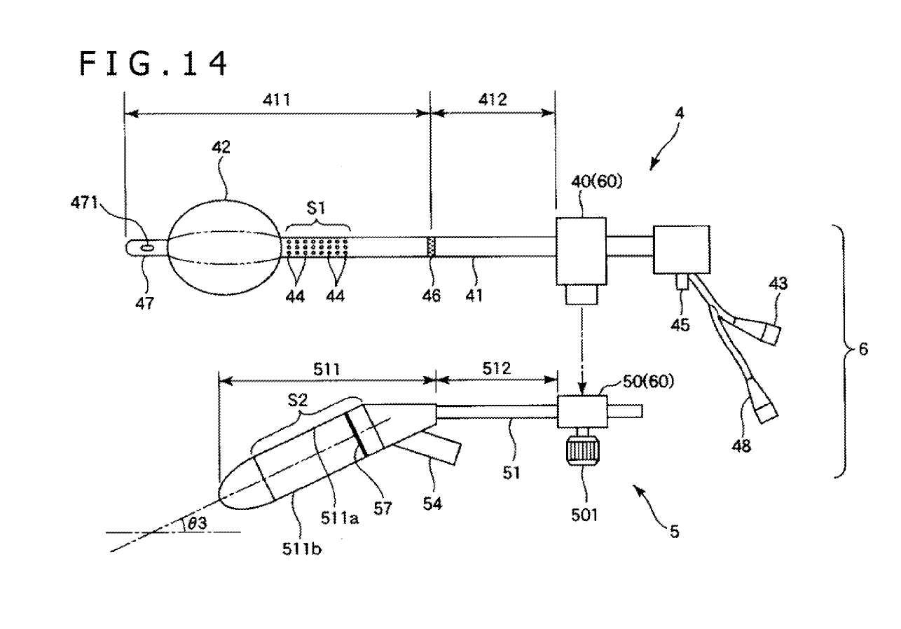

As shown in FIGS. 1 and 14, the insertion instrument 6 can include a urethral insertion portion (second insertion portion) 41 to be inserted into a urethra, a vaginal insertion portion (first insertion portion) 51 to be inserted into a vagina, and a support portion 60 supporting the urethral insertion portion 41 and the vaginal insertion portion 51. As disclosed above, the insertion instrument 6 can be composed essentially of the urethral insertion member 4 and the vaginal insertion member 5. The urethral insertion member 4 can have the urethral insertion portion 41, and the vaginal insertion member 5 has the vaginal insertion portion 51. In addition, the support portion 60 can include a support portion 40, which is possessed by the urethral insertion member 4 and supports the urethral insertion portion 41, and a support portion 50, which is possessed by the vaginal insertion member 5 and supports the vaginal insertion portion 51. In the insertion instrument 6, the urethral insertion member 4 and the vaginal insertion member 5 can be freely detachable by way of the support portions 40 and 50, respectively. The urethral insertion member 4 and the vaginal insertion member 5 will be sequentially described below.

The urethral insertion member 4 can include the elongated urethral insertion portion 41 whose portion ranging from a distal end to an intermediate portion of insertion portion 41 is to be inserted into a urethra, and the support portion 40 which supports the urethral insertion portion 41. In the following, for convenience of description, that portion of the urethral insertion member 4 which is located inside the urethra (inclusive of a bladder) in the mounted state will be referred to also as “insertion portion 411,” whereas that portion of the urethral insertion member 4 which is exposed via a urethra orifice to the outside of the body in the mounted state and which ranges to the support portion 40 will be referred to also as “non-insertion portion 412.”

The urethral insertion portion 41 can be in the shape of a tube with its distal end rounded. In addition, the insertion portion 411 is provided at its distal portion with an inflatable and deflatable balloon 42 and a urine drain portion 47. The balloon 42 functions as a restriction portion restricting the position in an axial direction of the urethral insertion member 4 in the inside of the urethra. For example, when the puncture apparatus 1 is used, the balloon 42 is inflated after inserted into a patient's bladder. Then, with the balloon 42 caught on a bladder neck, the position of the urethral insertion member 4 relative to the bladder and the urethra is fixed. In accordance with an exemplary embodiment, the urine drain portion 47 can be used for draining urine present inside the bladder.

The balloon 42 extends through the inside of the urethral insertion portion 41, to be connected to a balloon port 43 provided at a proximal portion of the urethral insertion portion 41. A balloon-inflating instrument such as a syringe can be connected to the balloon port 43. When a working fluid (a liquid such as physiological salt solution, or a gas or the like) is supplied from the balloon-inflating instrument into the balloon 42, the balloon 42 is inflated. When the working fluid is drawn out of the balloon 42 by the balloon-inflating instrument, the balloon 42 is deflated. In FIG. 14, the balloon 42 in its deflated state is drawn in a two-dot chain line, whereas the balloon 42 in its inflated state is drawn in a solid line.

The urine drain portion 47 can be provided with a drain hole 471 providing communication between an inside and an outside of the urine drain portion 47. In addition, the urine drain portion 47 can extend through the inside of the urethral insertion portion 41, to be connected to a urine drain port 48 provided at a proximal portion of the urethral insertion portion 41. Therefore, the urine introduced through the drain hole 471 into the urine drain portion 47 can be drained via the urine drain port 48.

The balloon 42 and the urine drain portion 47 can be configured by use of a double lumen, for example.

In addition, the insertion portion 411 can be formed with a plurality of suction holes 44 at an intermediate portion of the insertion portion 411. The plurality of suction holes 44 can be laid out over the whole range in the circumferential direction of the urethral insertion portion 41. Each of the suction holes 44 can be connected to a suction port 45 provided at a proximal portion of the urethral insertion portion 41, via the inside of the urethral insertion portion 41. A suction device such as a pump can be connected to the suction port 45. When the suction device is operated in a state wherein the urethral insertion portion 41 is inserted in the urethra, a urethral wall can be sucked and fixed onto the urethral insertion portion 41. When the urethral insertion portion 41 is pushed in toward the distal end (toward the inside of the body) under this condition, the urethra is also pushed in together with the urethral insertion portion 41. As a result, for example, the bladder can be shifted to such a position as not to overlap with a puncture route for the puncture member 3, whereby the puncture route for the puncture member 3 can be secured. Therefore, puncturing by the puncture member 3 can be carried out relatively accurately and safely. It is to be noted that the number of the suction holes 44 is not limited, for example, the number may be one. In addition, layout of the suction holes 44 is not limited, for example, the suction holes 44 may be formed in only a part of the range in the circumferential direction of the urethral insertion portion 41.

In addition, at the boundary portion between the insertion portion 411 and the non-insertion portion 412, a marker 46 can be provided to check the depth of insertion of the urethral insertion portion 41 into the urethra. When the urethral insertion portion 41 is inserted in the urethra and the balloon 42 is located inside the bladder, the marker 46 is located at the urethral orifice, which permits relatively easy checking of the depth of insertion of the insertion portion 411 into the urethra. The marker 46 is necessary only to be externally visible, and can be composed essentially of, for example, a colored portion, a recessed and projected portion, or the like. In accordance with an exemplary embodiment, a graduation with indications of distance from the distal end of the urethral insertion portion 41 may be provided, in place of the marker 46.

The length of the insertion portion 411 is not limited, and may be set, as necessary, according to the length of the urethra and the shape of the bladder of the patient, or the like. The length of the insertion portion 411 can be, for example, about 50 to 100 mm, in view of the fact that the length of a female urethra is generally about 30 to 50 mm.

The length of the non-insertion portion 412 (the spacing between the urethral orifice and the support portion 40) is not limited. The length can be, for example, not more than about 100 mm, preferably in the range of about 20 to 50 mm. By such a setting, the length of the non-insertion portion 412 can be made appropriate, which can provide relatively enhanced operability. For example, if the length of the non-insertion portion 412 exceeds the just-mentioned upper limit, the center of gravity of the puncture apparatus 1 would, depending on the configuration of the frame 2 or the like factors, be largely deviated from the patient, possibly leading to a lowered stability of the puncture apparatus 1 in the mounted state.

The material constituting the urethral insertion member 4 is not limited. Examples of the material applicable here include various metallic materials such as stainless steels, aluminum, aluminum alloys, titanium, titanium alloys, etc. and various resin materials.

Here, an inclination angle θ2 of the plane f9 relative to the plane f2 orthogonal to the axis J2 of the urethral insertion portion 41 can be, for example, about 20 to 60 degrees, more preferably about 30 to 45 degrees, and further preferably about 35 to 40 degrees. The sheath main body 31 can be set indwelling in the body so that the angle formed between the plane f9 and a plane orthogonal to the axis of the urethra is, for example, about 20 to 60 degrees, more preferably about 30 to 45 degrees, and further preferably about 35 to 40 degrees. Such a setting can make it relatively easy to perform the puncturing by the puncture member 3 and to make shorter the distance of puncture by the puncture member 3.

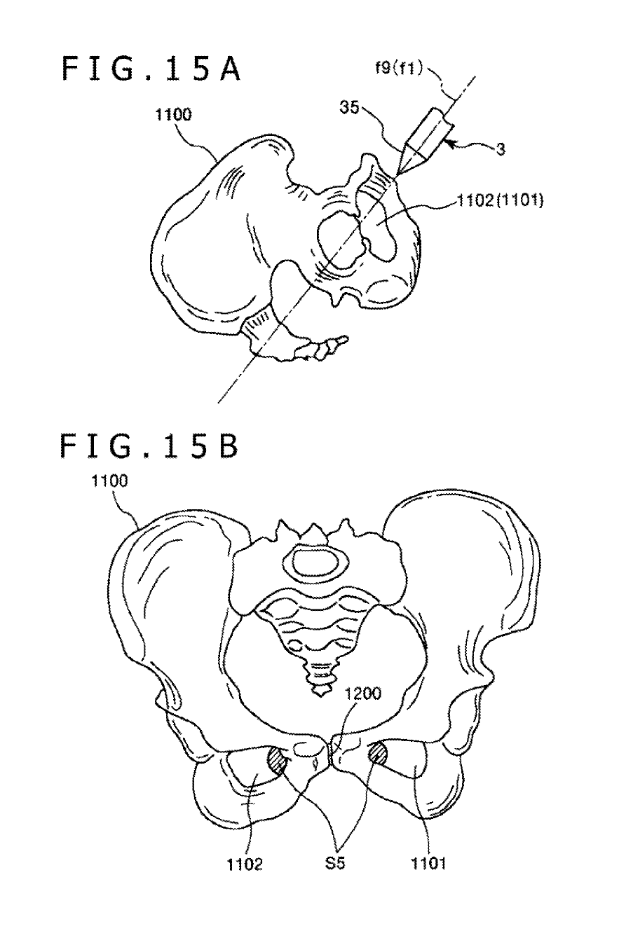

In accordance with an exemplary embodiment, setting the inclination angle θ2 to within the above-mentioned range can help ensure that the puncture member 3 can capture the left and right obturator foramens 1101 and 1102 of the pelvis 1100 wider on a planar basis, as shown in FIG. 15A, so that a wide puncturing space for the puncture member 3 can be secured. In a condition where a patient is put in a predetermined posture (lithotomy position), puncturing by the puncture member 3 can be performed in a direction comparatively nearer to a perpendicular direction relative to the obturator foramens 1101 and 1102. Therefore, the puncturing by the puncture member 3 can be carried out relatively easily. In addition, since the puncturing by the puncture member 3 is performed in a direction comparatively nearer to the perpendicular direction relative to the obturator foramens 1101 and 1102, it can help ensure that the puncture member 3 passes through a shallow part of tissue, so that the needle body 35 of the puncture member 3 can cross a region between the left and right obturator foramens 1101 and 1102 by passing a short distance. Accordingly, as shown in FIG. 15B, the puncture member 3 can be passed through those regions of the obturator foramens 1101 and 1102 which are near a pubic symphysis 1200, for example, through safety zones S5. Since the safety zones S5 are regions where there are few nerves and blood vessels the damage to which is to be obviated, puncturing by the puncture member 3 can be performed relatively safely. For example, the result can be minimal invasiveness, whereby burden on the patient can be suppressed. Thus, with the inclination angle θ2 set to within the above-mentioned range, puncturing of a patient by the puncture member 3 can be performed appropriately. In addition, by puncturing at the above-mentioned angle, it is facilitated to aim at a tissue between a middle-part urethra, which refers to an intermediate part in the lengthwise direction of the urethra and the vagina. The position between the middle-part urethra and the vagina is a position suitable as a site where to embed the implant 9 for treatment of urinary incontinence.

In the case where the inclination angle θ2 is less than the aforementioned lower limit or in excess of the aforementioned upper limit, there may arise an issue in that, depending on the individual differences concerning the patient or the posture during the procedure or the like factors, the puncture member 3 cannot capture the obturator foramens 1101 and 1102 wide on a planar basis or the puncture route cannot be made sufficiently short.

For example, the puncturing can be conducted in a condition where either one or both of the urethra and the vagina are positionally so shifted as to be pushed toward the inside of the body. Such an operation permits relatively easy puncturing of the tissue between the middle-part urethra and the vagina. The method for pushing in either one of the urethra and the vagina toward the inside of the body may be, for example, a method in which the urethral insertion member 4 and/or the vaginal insertion member 5 is inserted into an appropriate position, then, in this condition, the urethra and/or the vagina is sucked by the suction holes 44 and 59 (described later) possessed by these members 4 and 5, and thereafter the urethral insertion member 4 and/or the vaginal insertion member 5 is further moved toward the inside of the body along the axis thereof until reaching a predetermined position. In the condition where at least one of the urethra and the vagina has thus been positionally shifted so as to be pushed in toward the inside of the body, the sheath main body 31 is made to puncture the body perpendicularly to the left and right obturator foramens 1101 and 1102 of the pelvis, whereby a passage can be formed in a position suited to implanting of the implant 9.

For example, it can be preferable that a setting is made to cause an orbital path of the sheath main body 31 to pass the safety zones S5 of the left and right obturator foramens 1101 and 1102 of the pelvis, at least one of the urethra and the vagina is positionally shifted toward the inside of the body so as to locate the orbital path between the middle-part urethra and the vagina, and puncturing is performed along the orbital path of the main body 31, thereby forming the passage.

As shown in FIGS. 1 and 14, the vaginal insertion member 5 can include the elongated vaginal insertion portion (first insertion portion) 51 whose portion from a distal end to an intermediate portion of insertion portion 51 is inserted into a vagina, and the support portion 50 supporting the vaginal insertion portion 51. In the following, for convenience of description, that portion which is located in the vagina in the mounted state will be referred to also as “insertion portion 511,” and that portion which is exposed via a vaginal orifice to the outside of the body in the mounted state and which ranges to the support portion 50 will be referred to also as “non-insertion portion 512.”

The insertion portion 511 is elongated. In addition, the insertion portion 511 extends at an inclination relative to the insertion portion 411 so that the insertion portion 511 is spaced from the insertion portion 411 on the distal end. With the insertion portion 511 inclined relative to the insertion portion 411, a positional relationship between the insertion portions 411 and 511 can be set closer to the positional relationship between the urethra and the vagina, as compared with the case where the insertion portion 511 is not inclined in this way. In the mounted state, therefore, the puncture apparatus 1 is held stably onto the patient, and burden on the patient is mitigated. An inclination angle θ3 of the insertion portion 511 relative to the insertion portion 411 is not limited, for example, the inclination angle θ3 can be about 0 to 45 degrees, more preferably about 0 to 30 degrees, which can enable the above-disclosed effect to be conspicuously displayed. In the case where the inclination angle θ3 is less than the aforementioned lower limit or in excess of the aforementioned upper limit, there may arise an issue in that, depending on individual differences concerning the patient or the posture during the procedure or the like factors, the urethra and the vagina may be deformed unnaturally in the mounted state, possibly hampering the puncture apparatus 1 from being stably held.

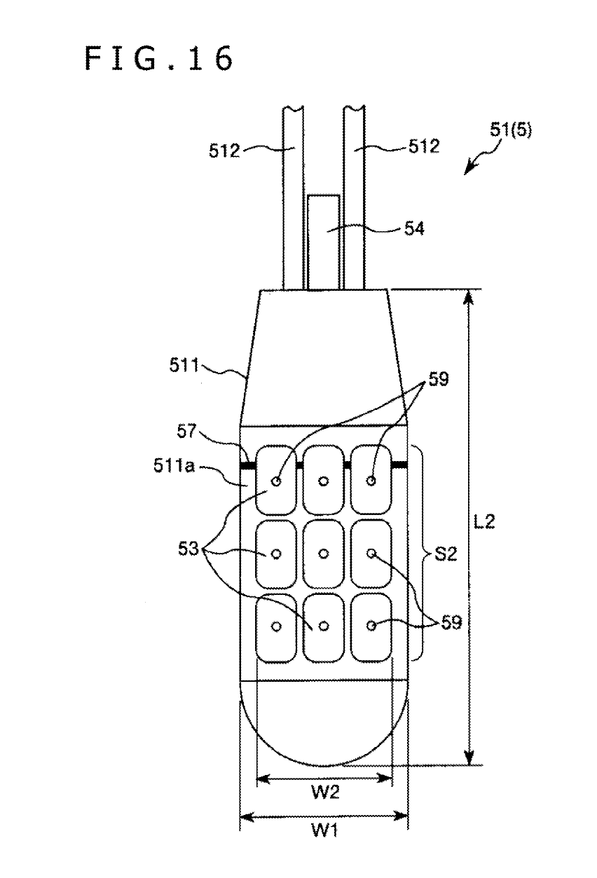

As shown in FIG. 16, the insertion portion 511 is in a flat shape collapsed in the vertical direction of the puncture apparatus 1 (in an array direction of the urethra and the vagina). In addition, the insertion portion 511 has a central portion having a substantially constant width and a somewhat rounded distal portion. A length L2 of the insertion portion 511 is not limited, and can be, for example, about 20 to 100 mm, more preferably about 30 to 60 mm. A width W1 of the insertion portion 511 is not limited, and can be, for example, about 10 to 40 mm, more preferably about 20 to 30 mm. In addition, the thickness of the insertion portion 511 is not limited, and can be, for example, about 5 to 25 mm, more preferably about 10 to 20 mm. Set to have such length, width, and thickness, the insertion portion 511 is suited in shape and size to ordinary vaginas. Therefore, stability of the puncture apparatus 1 in the mounted state is enhanced, and burden on the patient is relatively alleviated.

In addition, an upper surface (a surface on the urethral insertion portion 41 side) 511a of the insertion portion 511 is formed with a plurality of bottomed recesses 53. It is to be noted that the number of the recesses 53 is not limited, for example, the number may be one. In addition, each recess 53 can be provided with a single suction hole 59 in its bottom surface. Each suction hole 59 is connected to a suction port 54 provided at a proximal portion of the insertion portion 511, through the inside of the insertion portion 511. The suction port 54 is so provided as to be located in the outside of the living body in the mounted state. A suction device such as a pump can be connected to the suction port 54. When the suction device is operated in the condition where the insertion portion 511 is inserted in a vagina, an anterior wall of vagina, which is an upper surface of a vaginal wall, is sucked and fixed onto the insertion portion 511. When the vaginal insertion portion 51 with the vaginal wall sucked and fixed thereon is pushed toward the distal end (toward the inside of the body), the vaginal wall can be pushed in together with the vaginal insertion portion 51. Therefore, it is possible to put in good order the configuration and shape of the vaginal wall, to secure a puncture route for the puncture member 3, and to perform puncturing by the puncture member 3 relatively accurately and safely.

A region S2 in which the plurality of recesses 53 are formed is disposed opposite to a region S1. The needle tip of the puncture member 3 can pass between these regions S1 and S2. Since a lower surface of the urethral wall is sucked onto the insertion portion 411 in the region S1 and the anterior wall of vagina is sucked onto the insertion portion 511 in the region S2, as disclosed above, the urethral wall and the vaginal wall are spaced wider apart from each other between the regions S1 and S2. By causing the puncture member 3 to pass such a region, the puncturing by the puncture member 3 can be performed relatively safely.

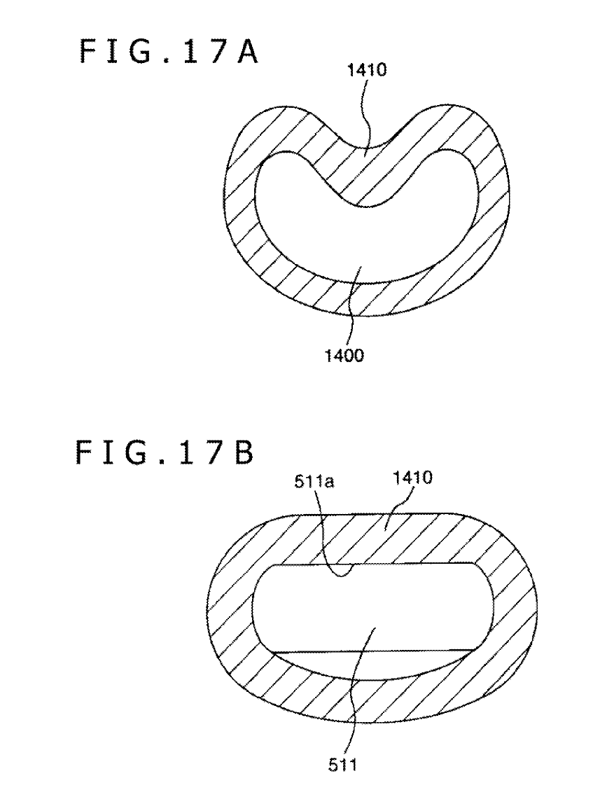

The region S2 ranges over substantially the whole region in the width direction of the upper surface 511a. A width W2 of the region S2 is not limited, and can be, for example, about 9 to 39 mm, more preferably about 19 to 29 mm, which can help enable the anterior wall of vagina to be sucked onto the insertion portion 511 relatively reliably, without being much influenced by the shape of the vaginal wall. For example, in some patients, a vagina 1400 may be so shaped that part of an anterior wall of vagina, 1410, is hanging down into the inside of the vagina, as shown in FIG. 17A. Even in such a case, setting the width W2 to within the above-mentioned range can help ensure that not only the hanging-down portion but also the portions on both sides of the hanging-down portion can be sucked in an assured manner, as shown in FIG. 17B. Therefore, the anterior wall of vagina can be spaced from the urethra relatively reliably, without being affected by the shape of the vagina. For example, in this exemplary embodiment, the insertion portion 511 is flat-shaped, so that the anterior wall of vagina can be so sucked as to be spaced apart from the urethra. Consequently, the biological tissue between the urethral wall and the vaginal wall can expand (spread).

In addition, the insertion portion 511 can be provided with a marker (puncture position checking portion) 57 with which a puncture route for the puncture apparatus 1 can be checked. For example, the puncture apparatus can be so fixed as to puncture a region (biological tissue) between the vaginal wall present on the upper side of the position where the marker 57 is located and the urethral wall. Therefore, operability and safety of the insertion instrument 6 can be enhanced. The marker 57 can be provided at least on a lower surface 511b of the insertion portion 511. The lower surface 511b is a surface, which is oriented toward the vaginal orifice can be visually confirmed by the operator via the vaginal orifice, in the mounted state. With the marker 57 provided on the lower surface 511b, therefore, the puncture route for the puncture apparatus 1 can be checked relatively reliably. In addition, the depth of insertion of the insertion portion 511 into the vagina can also be checked. It is to be noted that the marker 57 is necessary only to be externally visible, and can be configured by use of, for example, a colored portion, a recessed and projected portion, or the like.

The non-insertion portion 512 can be in the shape of a thin bar, which extends substantially in parallel to the urethral insertion portion 41. The spacing D between the non-insertion portion 512 and the urethral insertion portion 41 is not limited, and can be, for example, about 10 to 40 mm, correspondingly to the spacing between the urethral orifice and the vaginal orifice.

The length of the non-insertion portion 512 (the spacing between the vaginal orifice and the support portion 50) is not limited, and can be, for example, not more than about 100 mm, preferably in the range of about 20 to 50 mm, which can permit the non-insertion portion 512 to be appropriate in length, leading to relatively enhanced operability. If the length of the non-insertion portion 512 exceeds the just-mentioned upper limit, the center of gravity of the puncture apparatus 1 can, depending on the configuration of the frame 2 or the like factors, be largely deviated from the patient, possibly leading to a lowered stability of the puncture apparatus 1 in the mounted state.

The support portion 50 is provided with a male screw 501. With the male screw 501 fastened into a female screw (not shown) formed in the support portion 40, the support portions 40 and 50 can be fixed to each other.

The material constituting the vaginal insertion member 5 is not limited. Examples of the material applicable here include various metallic materials such as stainless steels, aluminum, aluminum alloys, titanium, titanium alloys, etc. and various resin materials, like the examples of the material for the urethral insertion member 4.

While the urethral insertion member 4 and the vaginal insertion member 5 constituting the insertion instrument 6 can be freely detachable in the puncture apparatus 1, this configuration is not limited. For example, a configuration may be adopted in which the urethral insertion member 4 and the vaginal insertion member 5 are non-detachable.

In addition, while the urethral insertion portion 41 can be fixed to the support portion 40 in the puncture apparatus 1, this configuration is not limited. For example, a configuration may be adopted wherein the urethral insertion portion 41 can be selectively switched between a state of being fixed to the support portion 40 and a state of being slidable in the axial direction relative to the support portion 40. For example, a configuration may be adopted wherein untightening of a screw provided on the support portion 40 permits the urethral insertion portion 41 to be slid relative to the support portion 40, whereas tightening the screw renders the urethral insertion portion 41 fixed to the support portion 40. This configuration can enable regulation of the length of the non-insertion portion 412, which can make the insertion instrument 6 relatively user-friendly. This can also apply to the vaginal insertion portion 51.

In addition, while the members can be fixed to the frame 2 so that the inclination angle θ2 will be constant in the puncture apparatus 1, this configuration is not limited. For example, the inclination angle θ2 may be variable, which can permit the inclination angle θ2 to be controlled according to the patient to be treated, which can improve the utility of the puncture apparatus 1.

The implant 9 is an embeddable (implantable) instrument for treatment of female urinary incontinence, for example, an instrument supporting the urethra 1300. For example, when the urethra 1300 tends to move toward the vagina 1400 side, the implant 9 can support the urethra 1300 in such a manner as to restrict its movement in a direction of coming away from the vagina 1400.

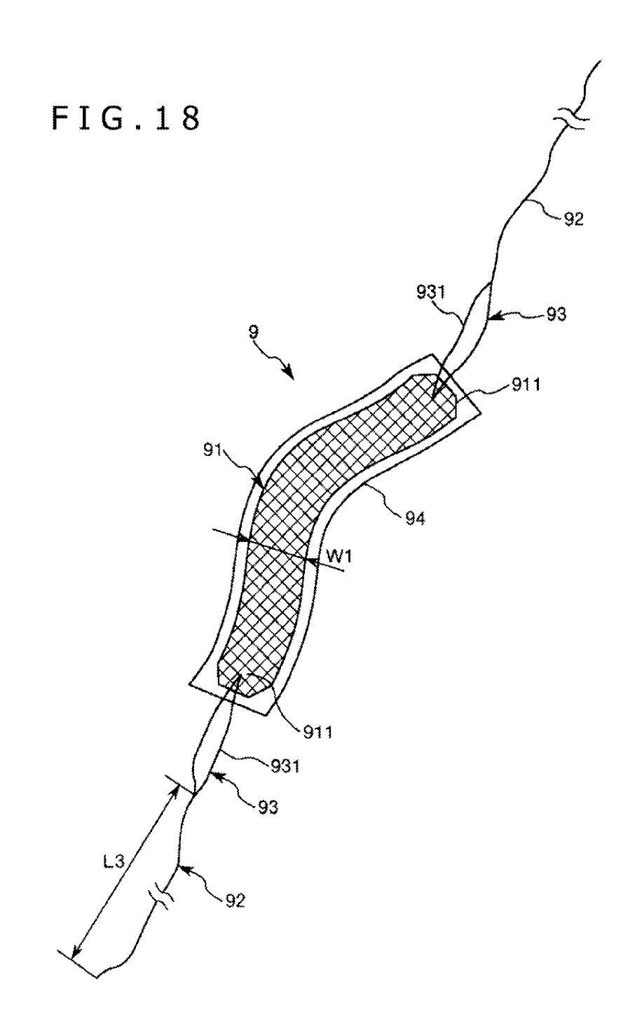

As shown in FIG. 18, the implant 9 can include the implant main body 91, guide portions 92, interlock portions 93 each interlocking the implant main body 91 and the guide portion 92 to each other, and a wrapping material 94 accommodating the implant main body 91.

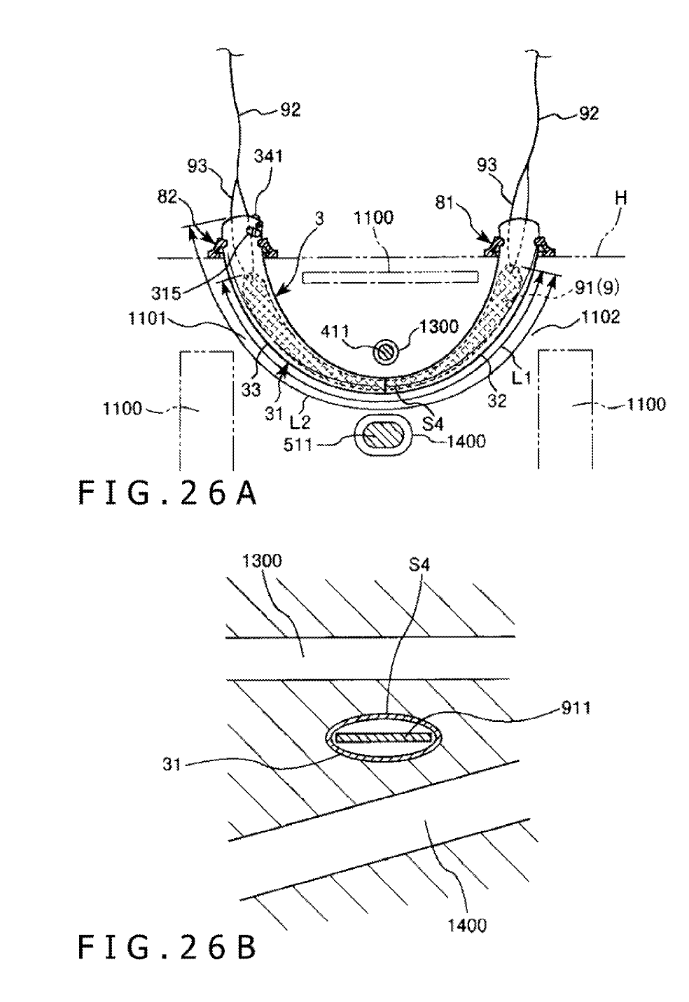

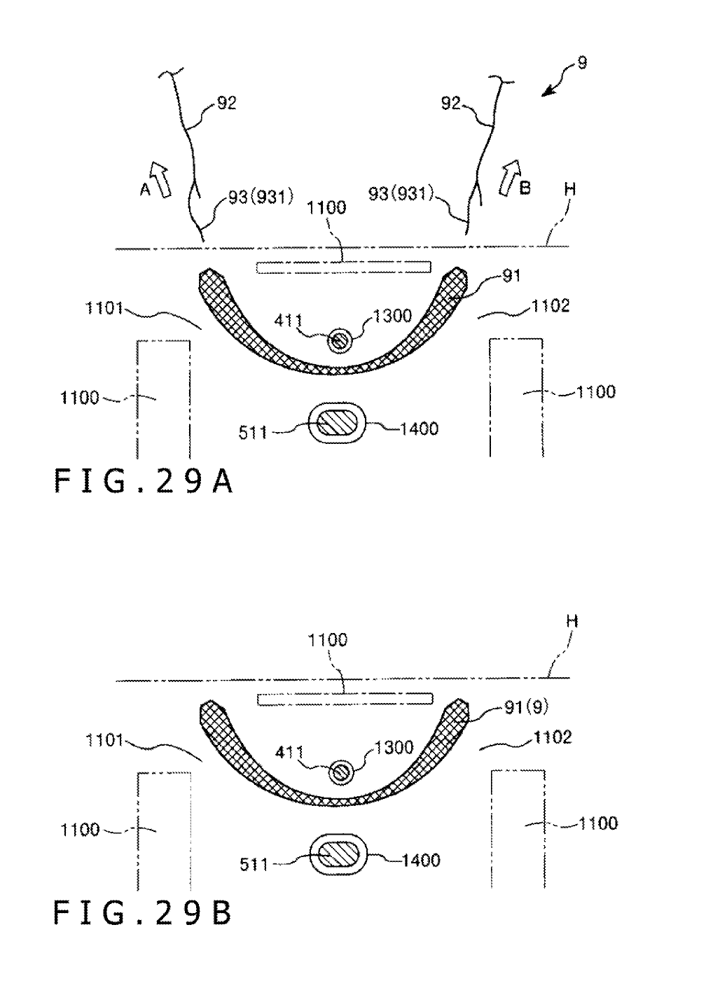

The implant main body 91 can be a portion which is placed indwelling in a living body, and, in its indwelling state, supports a urethra 1300 (see FIG. 29B). The implant main body 91 can be composed essentially of a band body configured by use of a mesh (network body), and is lower in rigidity than the sheath main body 31. A length L1 of the implant main body 91 is shorter than the length L2 of the sheath main body 31 (see FIG. 26A), which can help ensure that both ends of the implant main body 91 in the indwelling state are located at positions deviated in the depth direction from the body surface H, by a distance corresponding to the length by which the length L1 of the implant main body 91 is shorter than the length L2 of the sheath main body 31. Consequently, in the indwelling state, the implant main body 91 can avoid, as assuredly as possible, neurons that are generally present in large numbers in the vicinity of the body surface H, which can help ensure that the patient is less liable to feel pain in the indwelling state of the implant main body 91. Accordingly, the burden on the patient can be relatively mitigated, even in the case where the implant main body 91 is left indwelling for a comparatively long period of time.

In addition, when a pressure is externally exerted on that part of the body surface H which corresponds to the implant main body 91 set indwelling, transmission of the pressure to the implant main body 91 can be moderated, since a biological tissue is present between the body surface H and the implant main body 91. This can help ensure that movement of the implant main body 91 in the inside of the living body can be restrained. As a result, a pain attendant on a movement of the implant main body 91 can be relatively suppressed.

In addition, at both end portions of the implant main body 91, there can be formed decreasing-width portions 911 where the width of the implant main body 91 decreases, which can help ensure that formation of angular portions at the four corners of the implant main body 91 can be omitted. Therefore, for example at the time of inserting the implant main body 91 into the sheath main body 31, a part serving as a starting point bending or folding can be omitted. Consequently, the implant main body 91 can assume a posture of being assuredly spread out, in the indwelling state.

In addition, a linear body constituting the implant main body 91 is not limited. Examples of the linear body applicable here can include those that are circular in cross-sectional shape, and those, which are flat-shaped in cross section.

The guide portions 92 can be elongated in shape, can be provided on both end sides of the implant main body 91, and can each be interlocked to the implant main body 91 through the interlock portion 93. At the time of inserting the implant main body 91 into the sheath main body 31, the guide portion 92 can be inserted into the sheath main body 31 prior to the implant main body 91 and can guide the implant main body 91.

In addition, the guide portions 92 can be formed from a more rigid material as compared with the implant main body 91, and can be configured to be deformable into a curved shape. Here, the “rigid material” means a material which is elastic to such an extent that the guide portion 92 can be inserted into the sheath main body 31 and can be advanced by pushing as it is. When advanced in the sheath main body 31 by pushing, the guide portion 92 can be moved while being deformed along the curved shape of the sheath main body 31.

A length L3 of the guide portion 92 can be, for example, longer than the length L2 of the sheath main body 31, which can help ensure that while grasping an end portion of the guide portion 92 on the side of the implant main body 91, the guide portion 92 can be inserted into the sheath main body 31 via a one-side opening of the sheath main body 31, and can be made to protrude from the other-side opening of the sheath main body 31. In addition, by the simple operation of grasping the guide portion 92 protruding from the other-side opening and pulling the guide portion 92, the implant main body 91 can be guided and put into an inserted state.

The material constituting the guide portions 92 is not limited. Examples of the material applicable here can include various resin materials and metallic materials such as superelastic alloys (alloys that exhibit pseudoelasticity) represented by Ni—Ti alloys.

In addition, since the guide portions 92 can be provided on both sides of the implant main body 91, the implant main body 91 can be inserted into the sheath main body 31, starting from its end on either side of the implant 9. Consequently, the implant 9 can be inserted into the sheath main body 31 relatively swiftly.

In accordance with an exemplary embodiment, one of the two guide portions 92 may be omitted. In that case, for example, a string or a band or the like can be used in place of the one of the guide portions 92.

As shown in FIG. 18, the interlock portions 93 can be joined respectively to both end portions of the implant main body 91. Both the interlock portions 93 can have the same configuration; therefore, one of the interlock portions 93 will be described on a representative basis.

The interlock portion 93 can be composed essentially of a looped single cord 931. The cord 931 is passed through a network portion of the implant main body 91, and is non-fixedly interlocked with the linear material constituting the implant main body 91. In addition, the guide portion 92 is fixed to an intermediate portion of the cord 931. In accordance with an exemplary embodiment, a configuration may be adopted in which, for example, ring-shaped members are provided respectively at both end portions of the implant main body 91, and each cord 931 is passed through the ring-shaped member.

The method of fixing the interlock portion 93 and the guide portion 92 to each other is not limited. Examples of the applicable method include welding and adhesion by use of an adhesive. Further, the interlock portion 93 and the guide portion 92 may be formed integrally with each other. In this way, the implant main body 91 is interlocked to the guide portions 92.

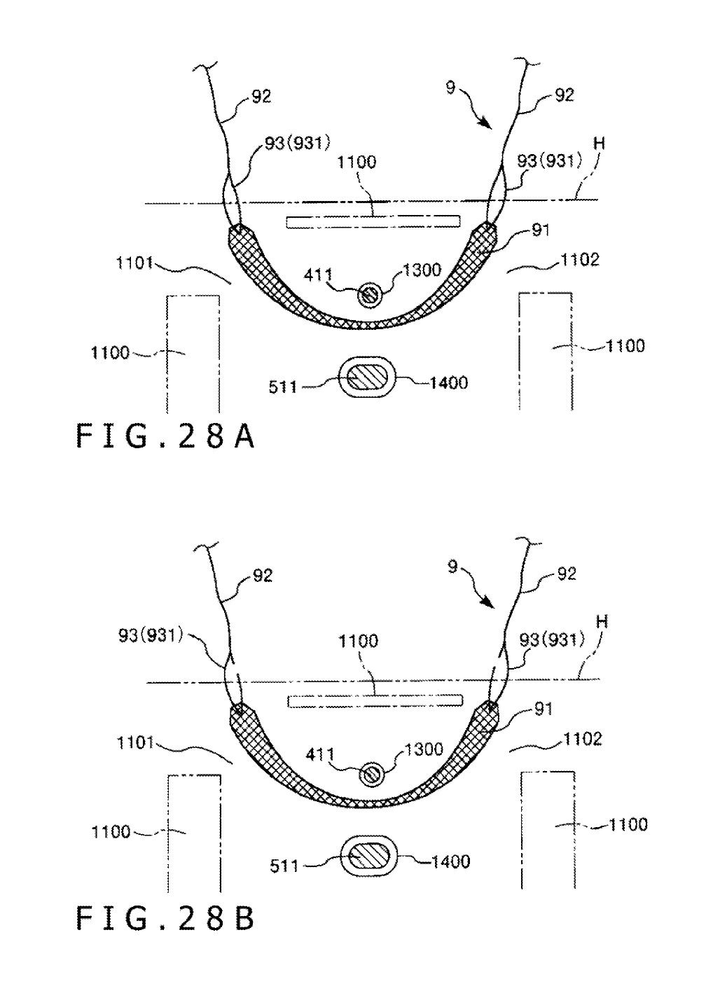

In addition, as shown in FIG. 28B, it is possible, by cutting the cords 931, to release the interlocking between the implant main body 91 and the guide portions 92 (hereinafter, this operation will be referred to as “releasing operation”).

In the inserted state in which the implant main body 91 is inserted in the sheath main body 31, the interlock portions 93 are protruding from the sheath main body 31 and exposed to the outside of the living body (see FIGS. 26A, 27A, 27B, 28A, and 28B), which helps enable the releasing operation to be performed outside of the living body. Therefore, it is possible to omit such an operation difficult to carry out as an operation of inserting the tips of a pair of scissors into the sheath main body 31 and cut the interlock portions 93. Accordingly, the releasing operation can be carried out relatively easily.

The wrapping material 94 can be bag-like in shape, and can accommodate the implant main body 91, which can help ensure that contamination of the implant main body 91 can be relatively prevented. It is to be noted that the wrapping material 94 is necessary only to be so sized as to be able to accommodate the implant main body 91, and may be so sized as to be able to accommodate the implant main body 91 and part of the interlock portions 93.

The respective materials constituting the implant main body 91, the interlock portions 93, and the wrapping material 94 are not limited. Examples of the materials applicable here can include various biocompatible resin materials, such as polypropylene, polyester, nylon, etc. and fibers.

The implant 9 and the sheath 30 as disclosed above can constitute an endopelvic treatment kit.

Now, an operation procedure of the puncture apparatus 1 and a method of placing the implant 9 indwelling according to the present disclosure will be disclosed.

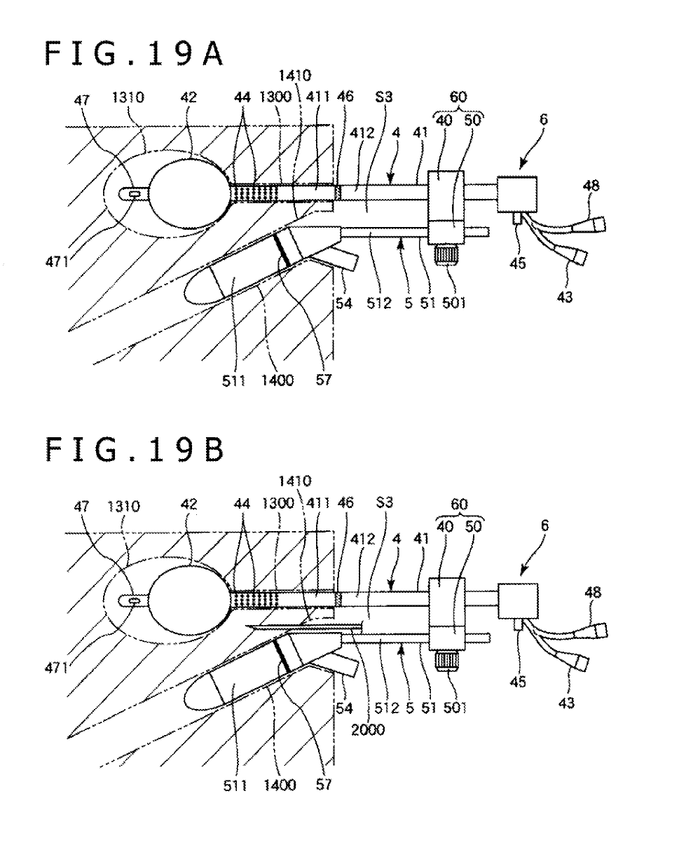

First, a patient is put in a lithotomy position on an operating table, and the insertion instrument 6 is mounted onto the patient, as shown in FIG. 19A. First, the urethral insertion portion 41 of the urethral insertion member 4 can be inserted into the patient's urethra 1300. In this instance, while checking the depth of insertion by observing the marker 46, the balloon 42 is disposed inside a bladder 1310. The urethra 1300 is rectified into a predetermined shape by the urethral insertion portion 41 having the predetermined shape. In the case of this exemplary embodiment, the urethra 1300 can be rectified into a rectilinear shape by the urethral insertion portion 41 having the rectilinear shape.

Next, the balloon 42 is inflated, and, if necessary, urine is drained from the inside of the bladder 1310 through the drain hole 471. In addition, the vaginal insertion portion 51 of the vaginal insertion member 5 is inserted into the patient's vagina 1400. In this instance, while checking the puncture position by observing the marker 57, the vaginal insertion portion 51 is inserted to an appropriate depth. Then, the male screw 501 is operated, to fix the support portions 40 and 50. By this, mounting of the insertion instrument 6 onto the patient is completed. In this state, the non-insertion portions 412 and 512 are spaced from each other, the support portion 60 is spaced from the body surface between the urethral orifice and the vaginal orifice, and that body surface is exposed. In the case where the insertion portion 511 and the anterior wall of vagina are spaced from each other and a gap (space) is formed therebetween, a space S3 causing a syringe to puncture the anterior wall of vagina or the like from the body surface between the urethral orifice and the vaginal orifice to a biological tissue between the urethra and the vagina can be formed.

Subsequently, suction devices are connected to the suction ports 45 and 54, and the suction devices are operated, whereby the urethra is sucked onto the urethral insertion portion 41, and the anterior wall of vagina is sucked onto the vaginal insertion portion 51. For example, when the urethra is properly sucked onto the urethral insertion portion 41, the suction holes 44 are closed with the urethral wall, so that the suction via the suction port 45 is stopped or weakened. Similarly, when the anterior wall of vagina is properly sucked onto the vaginal insertion portion 51, the suction holes 59 are closed with the vaginal wall, so that the suction via the suction port 54 is stopped or weakened. Therefore, based on the conditions of suction via the suction ports 45 and 54 (for example, a magnitude of sounds generated attendant on the suction via the suction ports 45 and 54), the operator can check whether or not the urethra and the anterior wall of vagina have been properly sucked onto the urethral insertion portion 41 and the vaginal insertion portion 51, respectively. In accordance with an exemplary embodiment, the insertion instrument 6 may be provided with a checking mechanism for mechanical checking of the sucked state. The checking mechanism is not limited, insofar as it enables checking of the sucked state. For example, the checking mechanism may be configured to include a flow measurement unit (negative-pressure sensor) measuring a flow rate through the suction port 54 and a decision unit deciding whether or not the sucking is being properly done, based on the measurement results sent from the flow measurement unit.

Next, humoral dissection is performed. As shown in FIG. 19B, a puncture needle of a syringe 2000 is caused to puncture the anterior wall of vagina, 1410, by way of the space (space S3) between the insertion portion 511 and the anterior wall of vagina, 1410, and a liquid such as physiological salt solution or local anesthetic is injected into the biological tissue between the urethra 1300 and the vagina 1400 (between the regions 51 and S2). As a result, the biological tissue between the regions S1 and S2 is expanded, the urethra is pressed against the urethral insertion portion 41, and the anterior wall of vagina, 1410, is pressed against the vaginal insertion portion 51.

Here, it can be preferable that the suction through the suction holes 44 and 59 is continuously conducted also during the humoral dissection. When the urethra is pressed against the urethral insertion portion 41 due to the humoral dissection, the urethra is further sucked onto the urethral insertion portion 41, so that the suction via the suction port 45 is stopped or weakened. Similarly, when the anterior wall of vagina is pressed against the vaginal insertion portion 51, the anterior wall of vagina is further sucked onto the vaginal insertion portion 51, so that the suction via the suction port 45 is stopped or weakened. Based on the conditions of suction via the suction ports 45 and 54, therefore, the operator can check whether or not the humoral dissection has been properly performed.

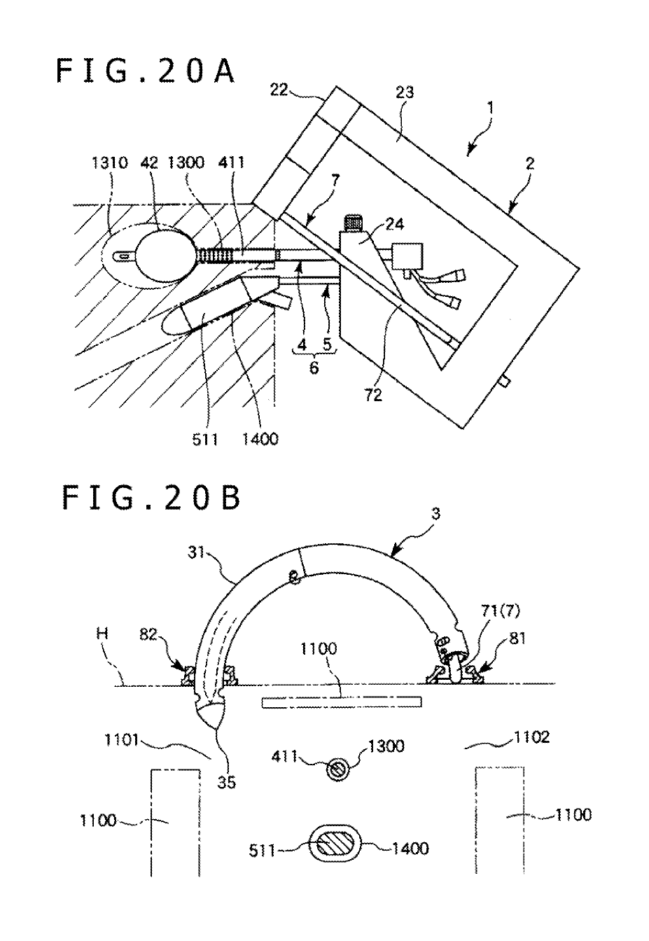

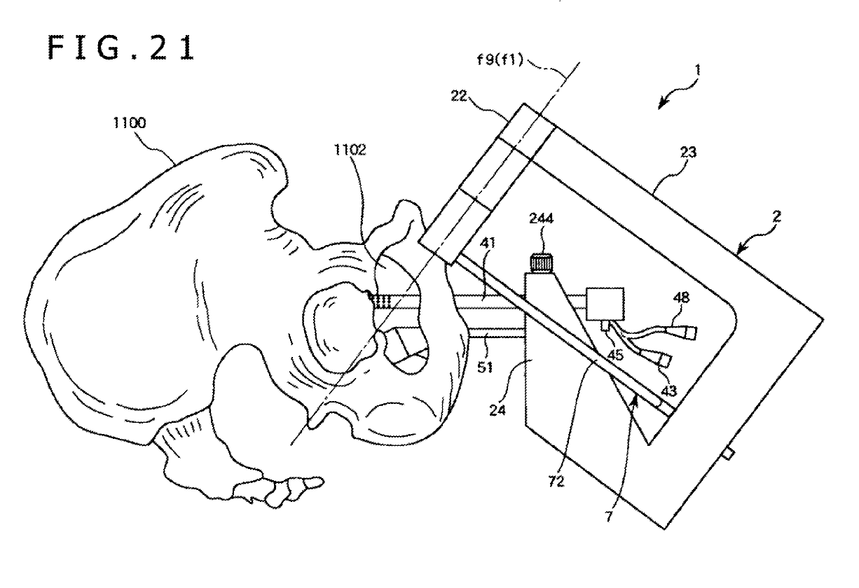

After the humoral dissection is conducted so that the urethra and the anterior wall of vagina are thereby sufficiently spaced from each other, the insertion instrument 6 is fixed to the frame 2, as shown in FIG. 20A. This results in that the puncture apparatus 1 is mounted on the patient. In this state, a positional relationship between the pelvis 1100 and the puncture apparatus 1 is as shown in FIG. 21.

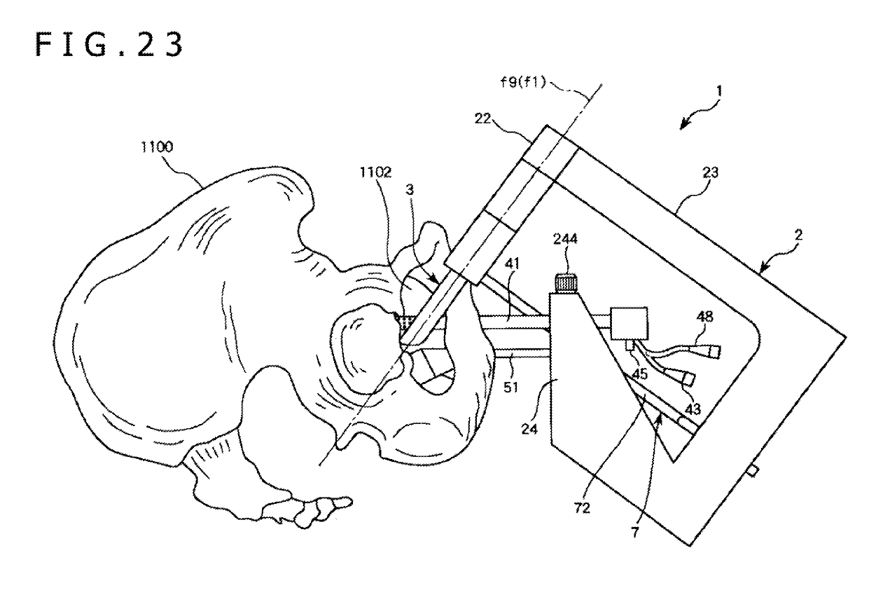

Next, for example, while grasping the interlock portion 23 of the frame 2 by one hand, the interlock portion 72 of the operating member 7 is grasped by the other hand, and the operating member 7 is rotated counterclockwise, as shown in FIG. 22A. This can help ensure that the needle body 35 of the puncture member 3 punctures the body surface H at the patient's right-hand inguinal region or a region in the vicinity thereof (first region) to enter the body, then sequentially pass an obturator foramen 1101 on one side, a position between the urethra 1300 and the vagina 1400, and an obturator foramen 1102 on the other side, thereafter protrudes from the body surface H at the patient's left-hand inguinal region or a region in the vicinity thereof (second region) to the outside of the body, and, finally, evacuates into the guide portion 22 (see FIG. 23).

As a result, the puncture member 3 is disposed in the living body, and, by the above disclosed principle, the anchors 81 and 82 are engaged with the sheath main body 31. Therefore, with the anchor 82 abutting on the body surface H, further insertion of the proximal portion of the sheath main body 31 into the living body is restricted. Thus, a state in which the proximal end of the sheath main body 31 is exposed to the outside of the living body can be relatively secured.

Subsequently, the operating member 7 is rotated clockwise in FIG. 22A. In this instance, the puncture member 3 also tends to rotate counterclockwise together with the operating member 7, but the abutment of the anchor 81 on the body surface H prevents a further rotation (movement). Therefore, the insertion portion 71 is pulled out of the puncture member 3 and the living body, while the state in which the distal end of the sheath main body 31 is exposed outside of the living body is maintained. Next, the puncture apparatus 1 (the members other than the puncture member 3) is detached from the patient, and, further, the needle body 35 is detached from the sheath main body 31. This results in a state in which only the sheath main body 31 is disposed inside the living body, as shown in FIG. 22B. The sheath main body 31 is disposed inside the living body, with both its distal end opening and its proximal end opening exposed outside of the living body.

Next, if necessary, the position of the sheath main body 31 can be put in good order. For example, the sheath main body 31 can be shifted toward the proximal end or the distal end so that the positions of the anchors 81 and 82 relative to the living body will be in left-right symmetry, which can help enable the central portion S4 of the sheath main body 31 to be reliably located between the urethra 1300 and the vagina 1400. In this state, as shown in FIG. 24, the central portion S4 is disposed with its width direction (the direction of the major axis J32) W substantially in parallel to the urethra 1300. The urethra 1300 rectified by the insertion of the urethral insertion member 4 and the width direction of the central portion S4 can be located substantially in parallel.

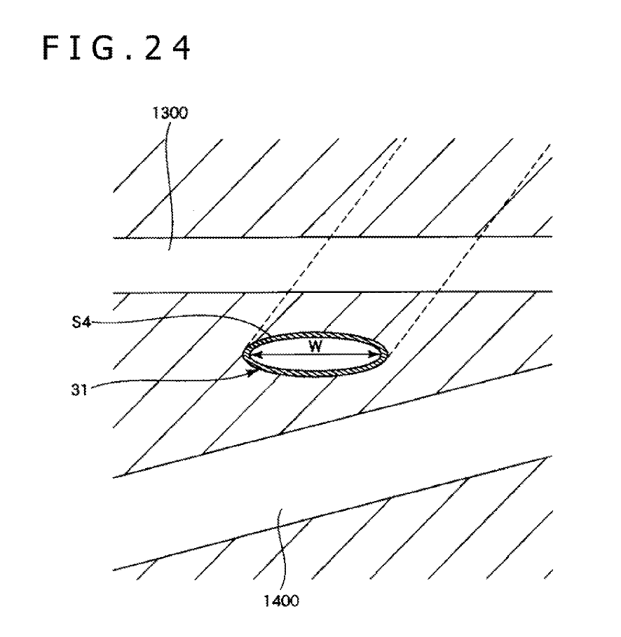

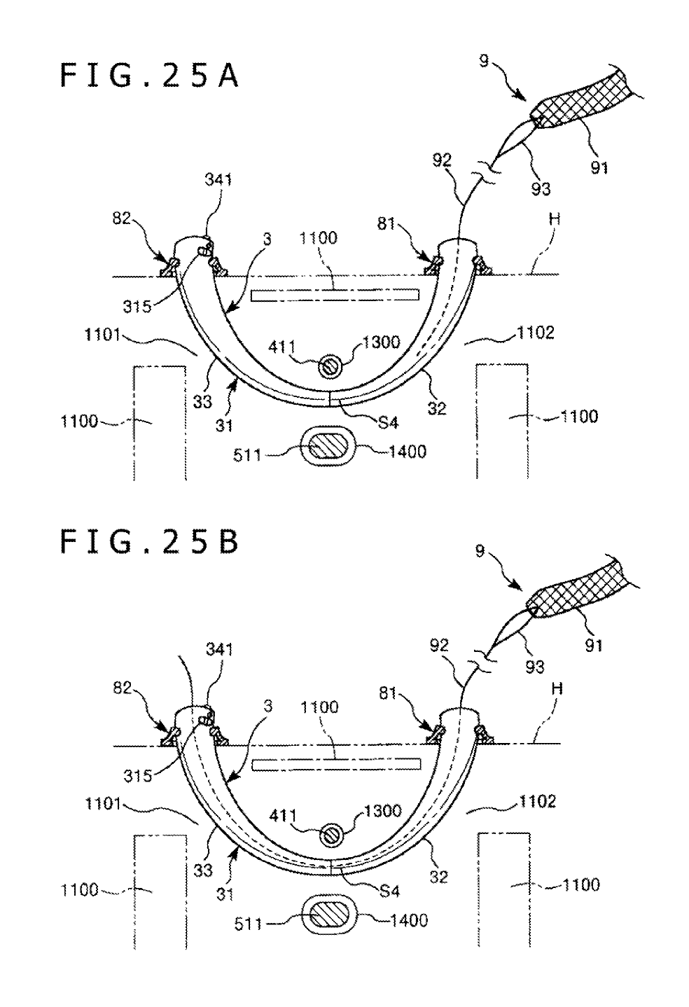

Subsequently, as shown in FIG. 25A, while taking the implant main body 91 out of the wrapping material 94, an end portion of the guide portion 92 on the side of the implant main body 91 is grasped, and the guide portion 92 is inserted into the sheath main body 31 via the proximal end opening of the sheath main body 31. When the guide portion 92 is further advanced by pushing, the guide portion 92 is inserted into the sheath main body 31 while being deformed along the curved shape of the sheath main body 31, to protrude from the distal end opening of the sheath main body 31 (see FIG. 25B).

Next, that part of the guide portion 92 which is protruding from the distal end opening of the sheath main body 31 is grasped by fingers or the like and is pulled. As a result, the guide portion 92 is pulled out via the distal end opening of the sheath main body 31, and the implant main body 91 is inserted into the sheath main body 31, resulting in the inserted state (see FIG. 26A). Since the sheath main body 31 is flat-shaped as aforementioned, the posture of the implant main body 91 in the inserted state follows the flat shape. Thus, the implant main body 91 is disposed in the sheath main body 31 in such a manner that its width direction coincides with the width direction of the sheath main body 31, as shown in FIG. 26B, which can help ensure that the implant main body 91 is disposed in parallel to the urethra 1300, which is in the rectified form.

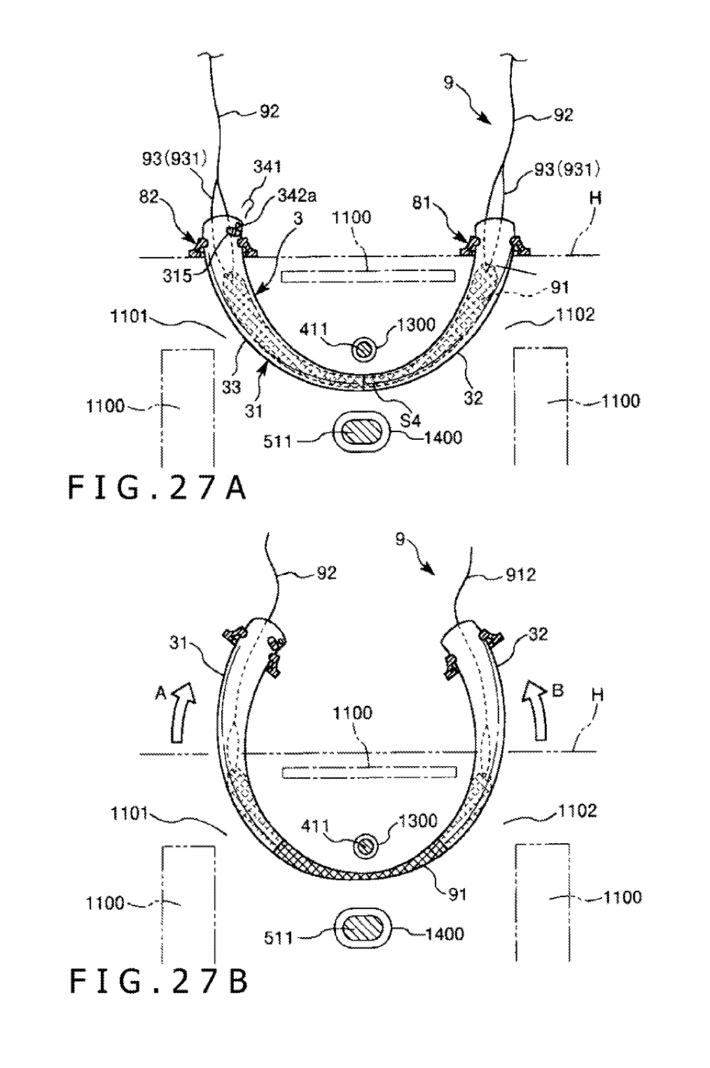

Subsequently, as shown in FIG. 27A, the string 341 exposed from the exposure holes 345 and 346 is cut. This can result in a state in which the sheath main body 31 can be split into the distal split piece 32 and the proximal split piece 33. In accordance with an exemplary embodiment, the exposure holes 345 and 346 can be located on the proximal end of the anchor 82, so that they are assuredly exposed outside of the living body. Accordingly, the string 341 can be cut relatively easily.

Next, sucking of the urethra by the urethral insertion portion 41 and the sucking of the anterior wall of vagina, 1410, by the vaginal insertion portion 51 are stopped. As a result, the positions and shapes of the urethra 1300 and the vagina 1400 are returned into the original positions and shapes in the natural state.

Subsequently, as shown in FIG. 27A, the connection between the distal split piece 32 and the proximal split piece 33 is released, then the distal split piece 32 is pulled distally out of the living body, and the proximal split piece 33 is pulled proximally out of the living body. In this instance, the distal split piece 32 and the proximal split piece 33 are substantially simultaneously moved in opposite directions, and the distal split piece 32 and the proximal split piece 33 are moved in arcuate courses along their own shapes (see FIG. 27B). As a result, the sheath main body 31 is smoothly removed from the living body. As the distal split piece 32 and the proximal split piece 33 are removed from the living body as aforementioned, the surrounding tissue having been forced spread or expanded by the sheath main body 31 returns into its original position, and the tissue comes into contact with the implant main body 91 gradually from a central portion toward both end portions of the implant main body 91. In accordance with an exemplary embodiment, the process in which the distal split piece 32 and the proximal split piece 33 are moved in directions along their shapes and the configuration in which the sheath main body 31 has the internal space permitting the implant main body 91 to move with a sufficiently low friction, as aforementioned, it helps ensure that no unnecessary tensile force is exerted on the implant main body 91 and that the implant main body 91 can be placed indwelling in an as-is state. This also helps eliminate the need for control of tension on the implant main body 91. As a result of the above disclosed operate, a state in which the implant 9 is set indwelling in the living body can be obtained, as shown in FIG. 28A.

With the sheath main body 31 removed from the living body by splitting it into the split pieces as aforementioned, the sheath main body 31 can be pulled out of the living body easily. In addition, since the sheath main body 31 can be pulled out of the living body without removing the anchors 81 and 82 from the sheath main body 31, the operation of pulling out the sheath main body 31 can be carried out relatively easily. In addition, according to such a pulling-out method, it can help ensure that the split pieces 32 and 33 being pulled out exert relatively little influence on the posture of the implant main body 91 in the region between the urethra 1300 and the vagina 1400.

In addition, since the split pieces 32 and 33 are pulled out in the state in which the urethral insertion member 4 is inserted in the urethra 1300, an excessive tension from being exerted on the urethra 1300 by the implant main body 91 placed indwelling in the living body can be relatively prevented.

Next, the urethral insertion member 4 is pulled out of the urethra 1300, and the vaginal insertion member 5 is pulled out of the vagina 1400. After the urethral insertion member 4 is pulled out, the urethra 1300 returns into its form in a natural state. In this case, since the implant main body 91 is embedded (implanted) in the biological tissue, the state in which the urethra 1300 in the natural state and the implant main body 91 are parallel can be maintained.

Subsequently, as shown in FIG. 28B, the releasing operation of cutting the interlock portions 93 of the implant 9 can be conducted. Since each interlock portion 93 is partly exposed outside of the living body, the releasing operation can be carried out relatively easily. After the cutting, as shown in FIG. 29A, the guide portions 92 are pulled respectively in a direction of arrow A and in a direction of arrow B in FIG. 29A, whereby the guide portions 92 and the interlock portions 93 can be detached from the implant main body 91, leaving the implant main body 91 in an indwelling state.

In accordance with an exemplary embodiment, since the cords 931 of the interlock portions 93 are non-fixedly interlocked with the linear body constituting the implant main body 91, an excessive tension from being exerted on the implant main body 91 when the cut cords 931 are pulled out can be relatively prevented.

As described above, according to the implant 9 of the present disclosure, the implant main body 91 in the indwelling state is located deep from the body surface H, which can help ensure that burden on the patient can be alleviated, even in the case where the implant main body 91 is left indwelling for a comparatively long period of time.

In addition, the use of the puncture apparatus 1 can help ensure that the operation placing the implant 9 indwelling can be dealt with only a less invasive procedure such as puncture by the puncture member 3, without need to perform a heavily invasive incision or the like. Therefore, relatively less the burden on the patient can be reduced and the procedure can be relatively safe. In addition, since the implant main body 91 can be implanted in parallel to the urethra 1300, the urethra 1300 can be supported in a wider region. In addition, the living body can be punctured by the puncture member 3 while avoiding the urethra 1300 and the vagina 1400, so that the puncture member 3 can be relatively prevented from puncturing the urethra 1300 or the vagina 1400, and thus, relatively safety can be secured. In addition, the issues encountered in the case of incision of a vagina, such as an issue that the implant 9 would be exposed to the inside of the vagina via a wound formed by the incision, or an issue of complications due to infection via the wound can be relatively prevented. Thus, the operation with the puncture apparatus 1 is relatively safe, and the implant 9 can be reliably implanted.

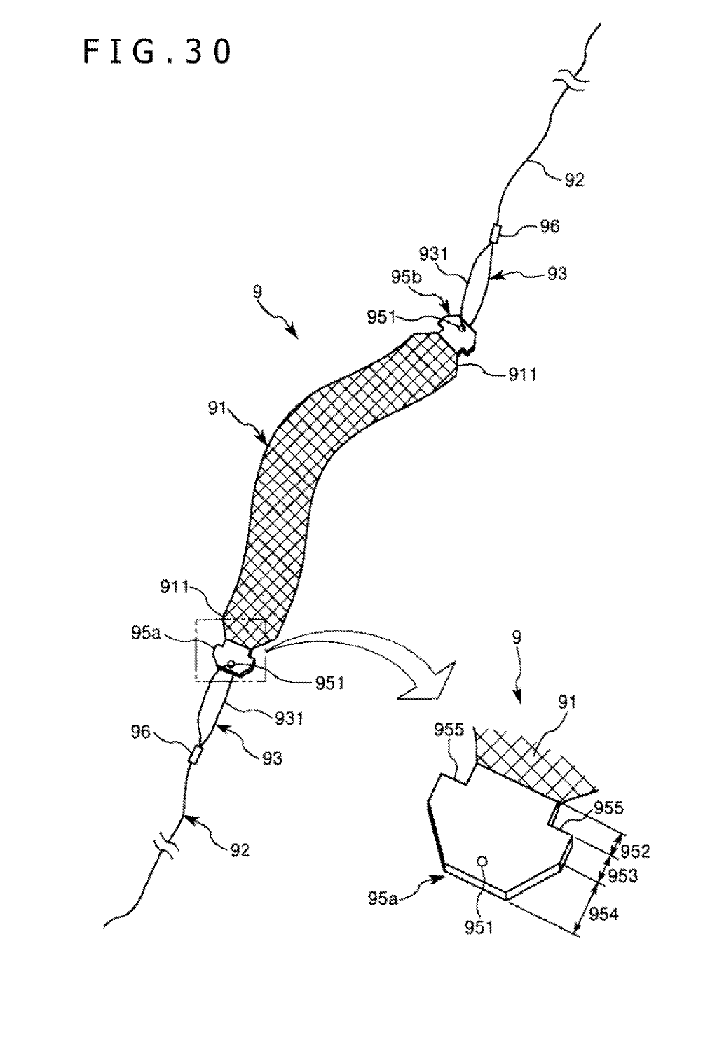

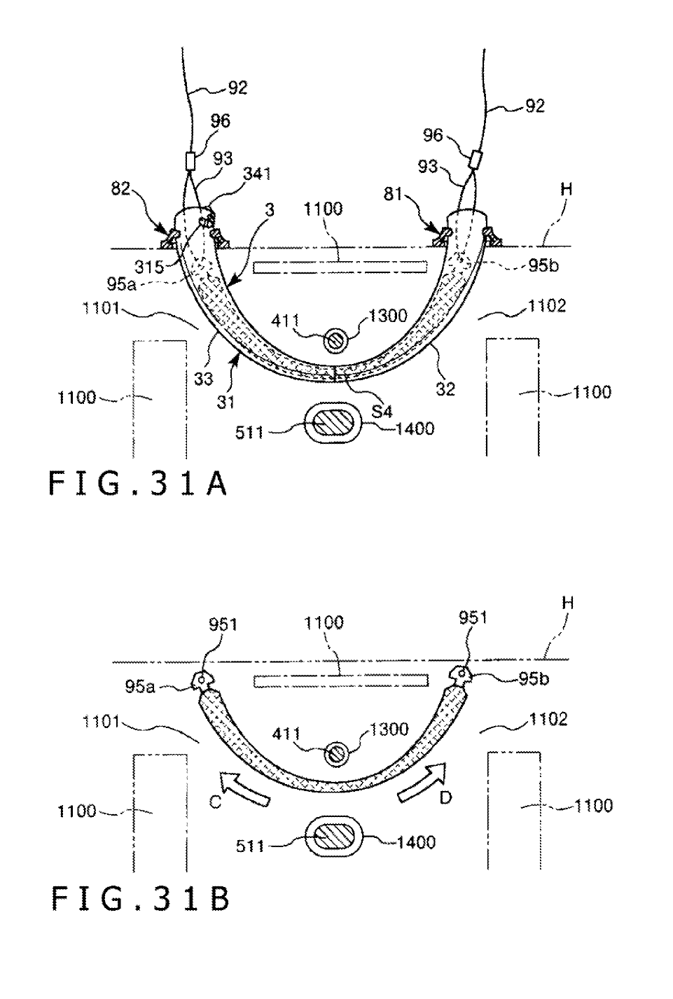

FIGS. 30 and 31 are views showing an implant according to a second exemplary embodiment of the present disclosure. Referring to these figures, the second exemplary embodiment of the implant and a method of placing an implant indwelling according to the present disclosure will be described below. The following description will center on differences from the above-described exemplary embodiment, and descriptions of the same items as those mentioned above will be omitted. This exemplary embodiment is the same as the first exemplary embodiment above, except for a difference in the configuration (shape) of the implant.

As shown in FIG. 30, an anchor portion 95a is provided on the lower side in FIG. 30 of an implant main body 91, and an anchor portion 95b is provided on the upper side in FIG. 30 of the implant main body 91.

The anchor portion 95a and the anchor portion 95b can be in the same configuration, and, therefore, the anchor 95a will be described on a representative basis.

The anchor portion 95a can be flat plate-like in shape, and can be formed from a material higher in rigidity than a material constituting a network portion of the implant main body 91. The maximum width of the anchor portion 95a can be smaller than the width of the network portion of the implant main body 91, which helps ensure that, for example, at the time of inserting the implant main body 91 into a wrapping material 94, the anchor portion 95a can be prevented from obstructing the inserting operation.

In addition, the anchor portion 95a can be divided into a small width portion 952 interlocked to the network portion, a large width portion 953 greater than the small width portion 952 in width, and a decreasing-width portion 954 where the width decreases along a direction toward the side opposite to the network portion. The small width portion 952, the large width portion 953, and the deceasing-width portion 954 are provided in this order from the side of the network portion of the implant main body 91.

At a boundary portion between the small width portion 952 and the large width portion 953, there can be formed a step portion 955 where the width of the anchor portion 95a can change abruptly, which can help permit the anchor portion 95a (step portion 955) to engage with a biological tissue in an indwelling state. As a result, the implant main body 91 is restricted in regard of movement in the direction of arrow D in FIG. 31B. In addition, by the anchor portion 95b provided on the side opposite to the anchor portion 95a, the implant main body 91 is restricted in regard of movement in the direction of arrow C in FIG. 31B.

Thus, according to the anchor portion 95a and the anchor portion 95b, the implant main body 91 in the indwelling state can be restricted in regard of movement in the longitudinal direction of implant main body 91. Therefore, the indwelling state of the implant main body 91 can be relatively maintained. Consequently, the implant main body 91 can support the urethra 1300 in a relatively stable manner.

In accordance with an exemplary embodiment, the anchor portion 95a and the anchor portion 95b may each be configured to increase also in thickness along a direction toward the network portion of the implant main body 91. This, in conjunction with the engagement of the step portion 955 with a biological tissue, can help enable the indwelling state to be reliably maintained.

In addition, each of the anchor portion 95a and the anchor portion 95b is formed with a through-hole 951 piercing itself in the thickness direction of the anchor portions 95a, 95b. A cord 931 of an interlock portion 93 is passed through the through-hole 951, whereby a guide portion 92 and the implant main body 91 are interlocked with each other.

In addition, each of the interlock portions 93 is provided, at its end portion on the side opposite to the implant main body 91, namely, at its portion exposed outside of the living body in the inserted state, with a marker 96 which can indicate the position of the implant main body 91.

Each of the markers 96 can be composed essentially of a colored portion, and can be so configured as to indicate the position of the implant main body 91 in the longitudinal direction thereof. In this exemplary embodiment, the markers 96 can be provided at positions substantially equidistant from the center of the implant main body 91 in the longitudinal direction, which can help ensure that by visually observing the markers 96 and controlling the positions of the markers 96 so that they are equally spaced from a body surface H, as shown in FIG. 31A. In accordance with an exemplary embodiment, a central portion of the implant main body 91 can be disposed into a position between a urethra 1300 and a vagina 1400 relatively easily and reliably. Consequently, the implant main body 91 can support the urethra 1300 in a relatively stable manner (see FIG. 31B).

Examples of the markers 96 can include those formed by coating with a paint (coating film), printing, dyeing, sticking of a sticker, a weld (fused body) or the like. In addition, the color of the markers 96 is not limited. Examples of the color applicable here can include various chromatic colors, achromatic colors, metallic colors, and fluorescent colors. The color of the markers 96 is necessary only to be different from the color of the interlock portions 93 (the surroundings of the marker 96).

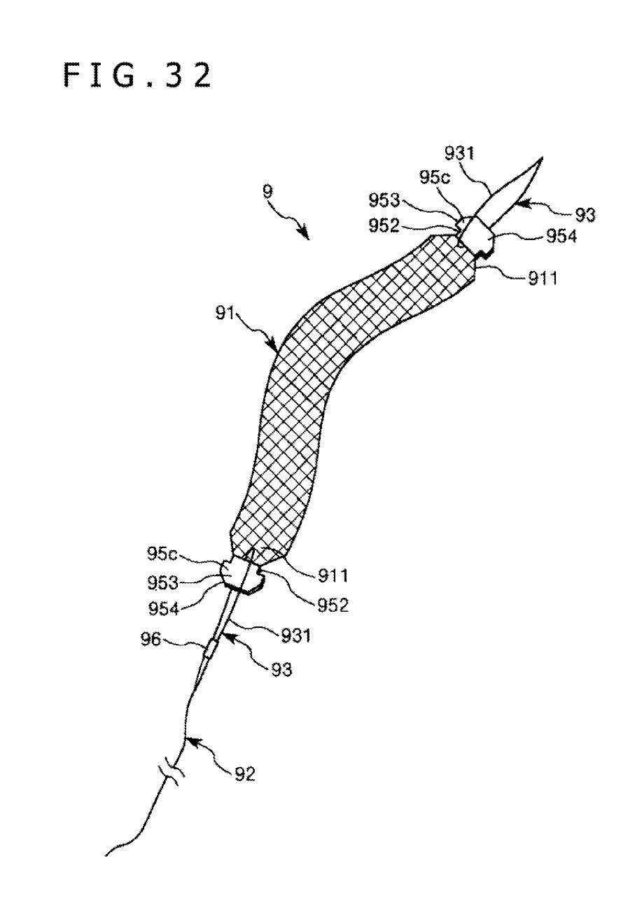

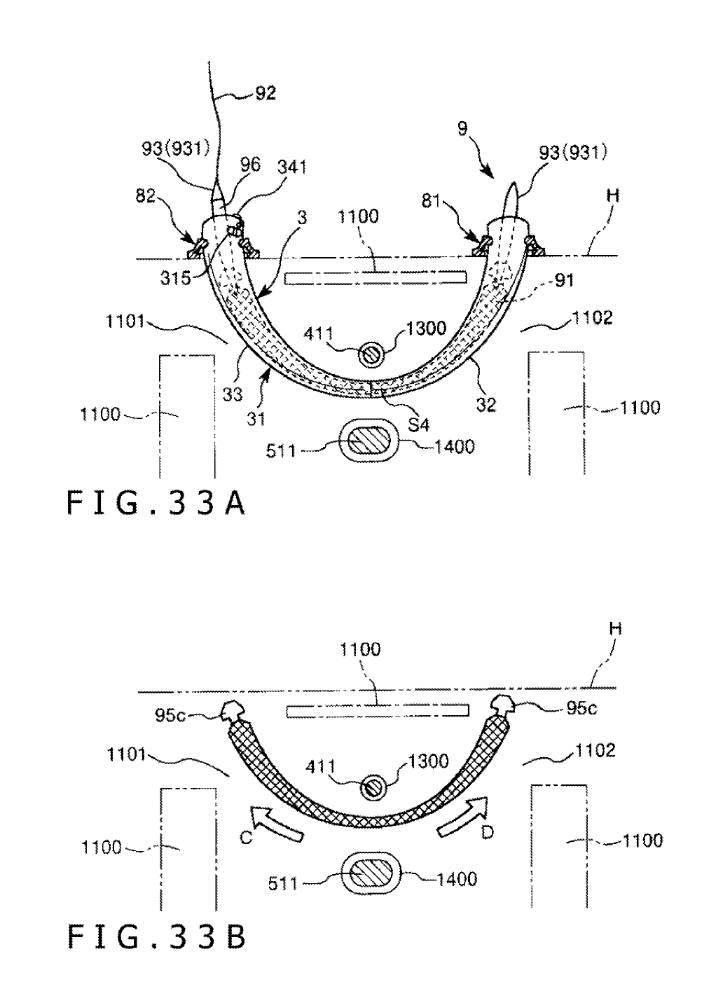

FIGS. 32 and 33 are views showing an implant according to a third exemplary embodiment of the present disclosure. Now, referring to these figures, the third exemplary embodiment of the implant and a method of placing an implant indwelling according to the present disclosure will be described below. The following description will center on differences from the above-described exemplary embodiments, and descriptions of the same items as those mentioned above will be omitted. This exemplary embodiment is the same as the second exemplary embodiment above, except for a difference in the configuration (shape) of the implant.

As shown in FIG. 32, at each anchor portion 95c, the aforementioned through-hole 951 can be omitted. The omission of the through-holes 951 can facilitate the formation of the anchor portions 95c, as compared with the formation of the anchor portion 95a and the anchor portion 95b. In addition, a cord 931 of each interlock portion 93 is passed through an opening of a network portion of an implant main body 91.

A single marker 96 can be provided at an intermediate portion of the interlock portion 93. In addition, as shown in FIG. 33A, the marker 96 can be disposed, with the distal end of a sheath main body 31 in an indwelling state as a yardstick, in such a manner that when the distance of the marker 96 from a body surface H is controlled to be equal to that of the distal end of the sheath main body 31, a central portion of the implant main body 91 is located between a urethra 1300 and a vagina 1400 (see FIG. 33B). Since comparison with the marker 96 is thus located in a position comparatively near the marker 96, an operation of controlling the position of the implant 9 can be carried out relatively easily and accurately.

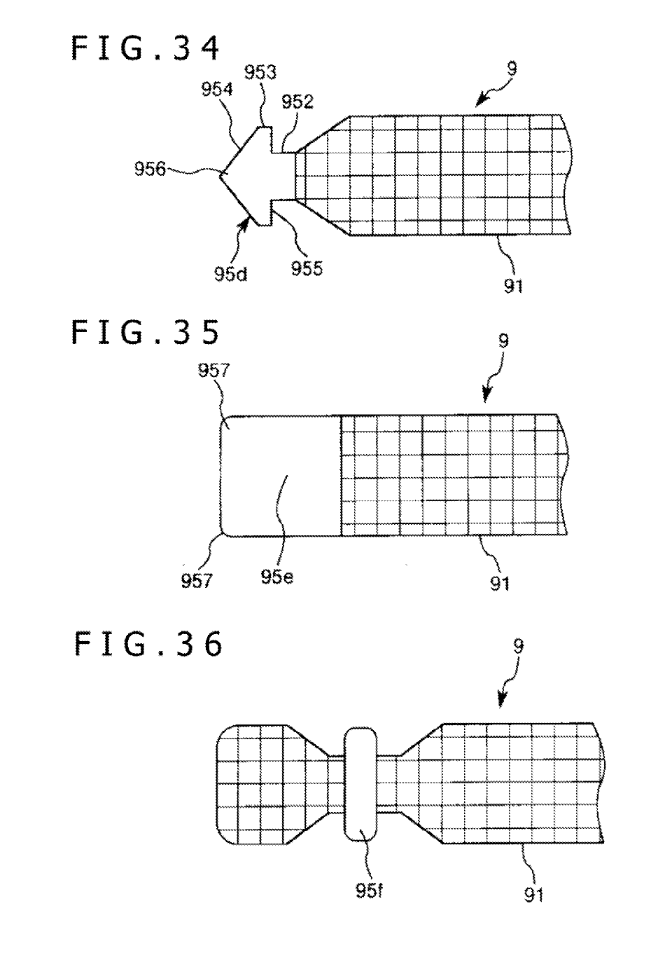

FIG. 34 is a plan view showing an implant according to a fourth exemplary embodiment of the present disclosure. Referring to this figure, the fourth exemplary embodiment of the implant and a method of placing an implant indwelling according to the present disclosure will be described below.

The following description will center on differences from the above-described exemplary embodiments, and descriptions of the same items as those mentioned above will be omitted. This exemplary embodiment is the same as the third exemplary embodiment above, except for a difference in the configuration (shape) of the anchor portion.

As shown in FIG. 34, a distal portion 956 of a decreasing-width portion 954 of an anchor portion 95d is tapered off into a sharp-pointed shape, which can help ensure that at the time of inserting an implant main body 91 into a wrapping material 94, the anchor portion 95d can be prevented from being caught on the wrapping material 94. Therefore, an operation of accommodating the implant main body 91 into the wrapping material 94 can be carried out relatively easily.

FIG. 35 is a plan view showing an implant according to a fifth exemplary embodiment of the present disclosure. Referring to this figure, the fifth exemplary embodiment of the implant and a method of placing an implant indwelling according to the present disclosure will be described below. The following description will center on differences from the above-described exemplary embodiment, and descriptions of the same items as mentioned above will be omitted.

This exemplary embodiment is the same as the fourth exemplary embodiment above, except for a difference in the configuration (shape) of the anchor portion.

As shown in FIG. 35, an anchor portion 95e is in a roughly rectangular shape in plan view. In addition, the anchor portion 95e is so shaped that its corner portions 957 on the distal end are somewhat rounded, which can help ensure that, in an indwelling state, the corner portions can be relatively prevented from damaging a biological tissue. In addition, the comparatively simple shape of the anchor portion 95e can promise excellent productivity.

FIG. 36 is a plan view showing an implant according to a sixth exemplary embodiment of the present disclosure. Referring to this figure, the sixth exemplary embodiment of the implant and a method of placing an implant indwelling according to the present disclosure will be described below. The following description will center on differences from the above-mentioned exemplary embodiments, and descriptions of the same items as mentioned above will be omitted.

This exemplary embodiment is the same as the fifth exemplary embodiment above, except for a difference in the configuration (shape) of the anchor portion.

As shown in FIG. 36, an anchor portion 95f is provided at an intermediate portion of an implant main body 91 in the longitudinal direction. In addition, the anchor portion 95f is ring-like in shape, which helps enable the anchor portion 95f, in an indwelling state, to engage with a biological tissue over the whole circumference in the circumferential direction. Accordingly, the indwelling state can be reliably maintained.

In addition, a network portion of the implant main body 91 is inserted into and fixed to the anchor portion 95f in the manner of being constricted in width. Such a configuration helps enable easier formation of an implant 9.

It is to be noted that the implant main body 91 may be formed to be partly lacking, according to an inside diameter of the anchor portion 95f. For example, the implant main body 91 may be constricted (necked) at a part in the longitudinal direction of the implant main body 91.

In addition, while openings of the anchor portion 95f are circular in this exemplary embodiment, they may be flat-shaped in conformity with the shape of the implant main body 91.

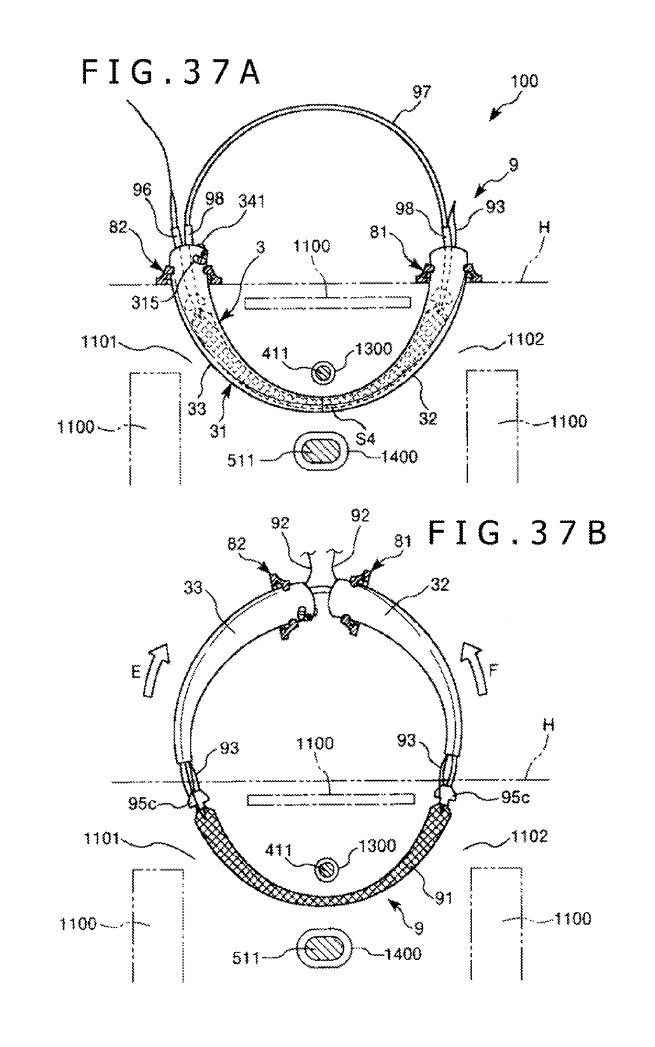

FIGS. 37A and 37B are views for illustrating a procedure of placing indwelling an implant according to a seventh exemplary embodiment of the present disclosure. Referring to these figures, the seventh exemplary embodiment of the implant and a method of placing an implant indwelling according to the present disclosure will be described below. The following description will center on differences from the above-described exemplary embodiments, and descriptions of the same items as those mentioned above will be omitted.

This exemplary embodiment is the same as the third exemplary embodiment above, except for a difference in the configuration (shape) of the endopelvic treatment kit.

In accordance with an exemplary embodiment, an endopelvic treatment kit having a guide member 97 is disclosed. The guide member 97 is elongated in shape, and is composed essentially of a rigid member, which is semi-arcuate in general shape. In addition, the guide member 97 is provided near both end portions thereof (at its intermediate portions in the longitudinal direction) with guide member-side markers 98. The guide member-side markers 98 are provided at positions roughly equidistant from a central portion of the guide member 97 in the longitudinal direction.

A method of using the guide member 97 configured as above will be described.

First, as shown in FIG. 37A, both end portions of the guide member 97 are inserted into sheath main body 31 via both end openings of the sheath main body 31, in an indwelling state, and are brought into abutment on anchor portions 95c, respectively, whereby positioning is conducted. In a method for this positioning, first, the distances of markers 96 of the anchor portions 95c from a body surface H or from both end portions of a sheath 30 are controlled to be approximately equal to each other. Next, the height of the markers 96 of the anchor portions 95c and the height of the guide member-side markers 98 are controlled to be equal to each other. By this, the guide member 97 can be positioned with relatively high accuracy.

Subsequently, as shown in FIG. 37B, while holding the guide member 97, and while maintaining its state of abutment on the anchor portions 95c, a sheath main body 31 is pulled out along the guide member 97. In this instance, a distal split piece 32 is pulled out along a direction of arrow E in FIG. 37B, and a proximal split piece 33 is pulled out along a direction of arrow F in FIG. 37B.

Then, the distal split piece 32, the proximal split piece 33 and the guide member 97 are grasped collectively, and the guide member 97 is pulled out of the living body.

Thus, the guide member 97 can be so configured that at the time of pulling out the distal split piece 32 and the proximal split piece 33, the guide member 97 is in abutment on the anchor portions 95c, which can help ensure that the implant main body 91 can be securely prevented from being caught on the distal split piece 32 or the proximal split piece 33 and moved together with the sheath main body 31. Furthermore, the implant main body 91 can be placed into an indwelling state by a simple operation of pulling out the distal split piece 32 or the proximal split piece 33 along the guide member 97.

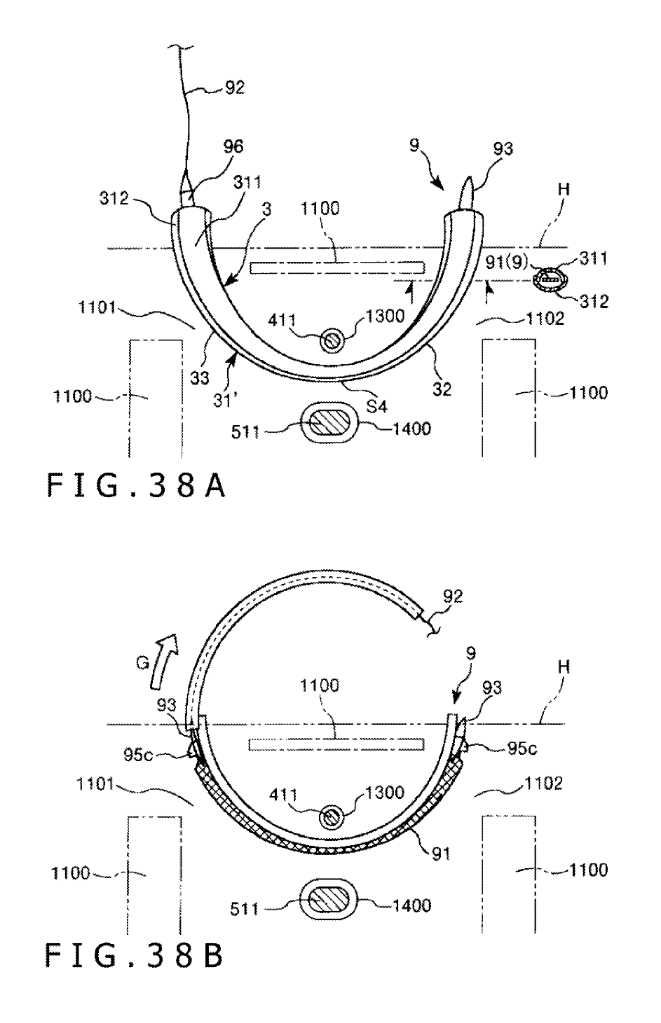

FIGS. 38A and 38B are views for illustrating a procedure of placing indwelling an implant according to an eighth exemplary embodiment of the present disclosure. Referring to these figures, the eighth exemplary embodiment of the implant and a method of placing an implant indwelling according to the present disclosure will be described below. The following description will center on differences from the above-described exemplary embodiments, and descriptions of the same items as those mentioned above will be omitted.

This exemplary embodiment is the same as the seventh exemplary embodiment above, except for a difference in the configuration (shape) of the sheath.

As shown in FIG. 38A, a sheath main body 31′ is composed essentially of an inside split piece 311 and an outside split piece 312. Each of the inside split piece 311 and the outside split piece 312 is C-shaped in cross section, and is semi-arcuate in general shape. In addition, the inside split piece 311 and the outside split piece 312 are interlocked to each other, with their both end portions in cross-sectional shape overlapping. In this interlocked state, in the cross-sectional shape of the sheath main body 31′, both end portions of the inside split piece 311 are located on the inside of the outside split piece 312. In addition, the inside split piece 311 and the outside split piece 312 are released from the interlocking, by shifting (sliding) them from each other along the longitudinal direction.

Starting from an inserted state shown in FIG. 38A, the outside split piece 312 is slid relative to the inside split piece 311 in a direction of arrow G in FIG. 38B, to be pulled out. In this instance, since the inside split piece 311 is located on the body surface H side of an implant main body 91, as shown in FIG. 38B, the implant main body 91 can be prevented from moving toward the body surface H side. This can help enable an operation of placing the implant main body 91 into an indwelling state to be performed accurately.

Then, the inside split piece 311 is pulled out, whereby the implant main body 91 can be placed in the indwelling state.

It is to be noted that while the outside split piece 312 is pulled out of a living body first in this exemplary embodiment, this is not limited, and the inside split piece 311 may be pulled out of the living body first.

In addition, the inside split piece 311 and the outside split piece 312 may each be split at a central portion thereof into smaller split pieces, which may be pulled out in opposite directions.

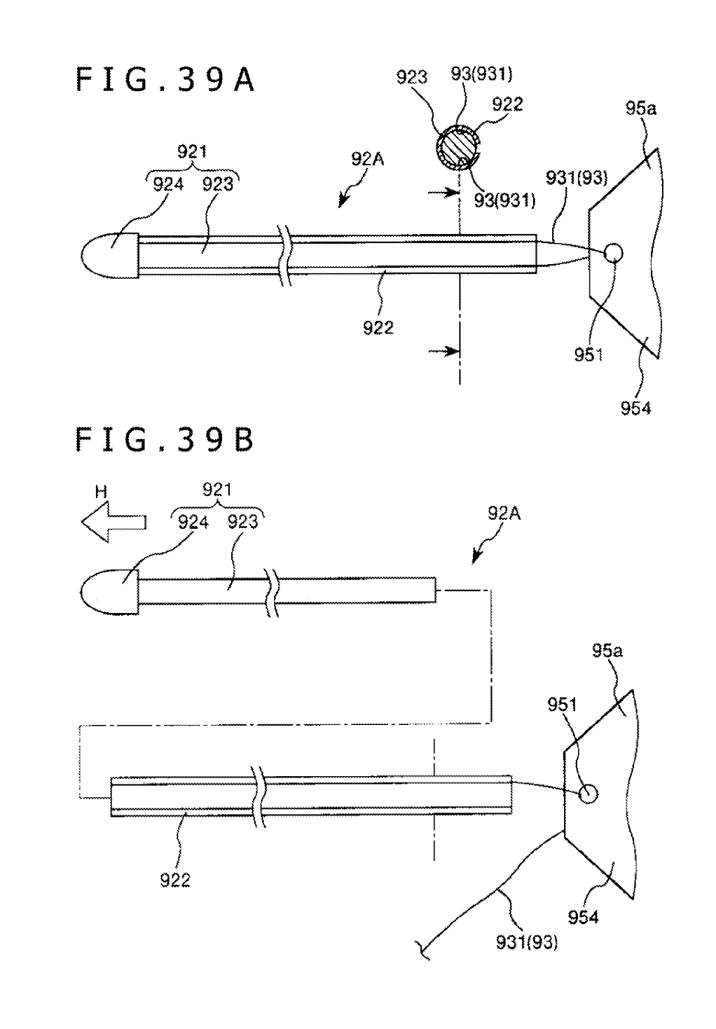

FIGS. 39A and 39B are views for illustrating a procedure of placing indwelling an implant according to a ninth exemplary embodiment of the present disclosure. Referring to these figures, the ninth exemplary embodiment of the implant and a method of placing an implant indwelling according to the present disclosure will be described below. The following description will center on differences from the above-described exemplary embodiments, and descriptions of the same items as those mentioned above will be omitted.

This exemplary embodiment is the same as the second exemplary embodiment above, except for a difference in the configuration (shape) of the guide portion.

As shown in FIG. 39A, a guide portion 92A is composed essentially of a linear body 921, and a tubular body 922 in which to insert the linear body 921.

The linear body 921 can be divided into a small outside diameter portion 923 which is elongated in shape, and a large outside diameter portion 924 which is provided on a distal end of the small outside diameter portion 923 and is greater than the small outside diameter portion 923 in outside diameter. The outside diameter of the small outside diameter portion 923 is approximately equal to an inside diameter of the tubular body 922. In addition, the large outside diameter portion 924 abuts on an end portion of a distal opening of the tubular body 922, thereby being inhibited from insertion into the tubular body 922.

A cord 931 of an interlock portion 93 is inserted into the tubular body 922 from the proximal end, and, in this state, the linear body 921 is inserted into the tubular body 922 from the distal end, whereby the cord 931 is clamped between the tubular body 922 and the linear body 921. As a result, the guide portion 92A and an implant main body 91 are fixed and interlocked to each other.

In addition, as shown in FIG. 39B, while grasping the large outside diameter portion 924, the linear body 921 is pulled out in the direction of arrow H, whereby the interlocking between the guide portion 92A and the implant main body 91 can be easily released (canceled).

According to the guide portion 92A configured as above, an operation of cutting an interlock portion 93, which might otherwise be needed in the releasing operation, can be omitted. Consequently, the releasing operation can be carried out relatively easily.

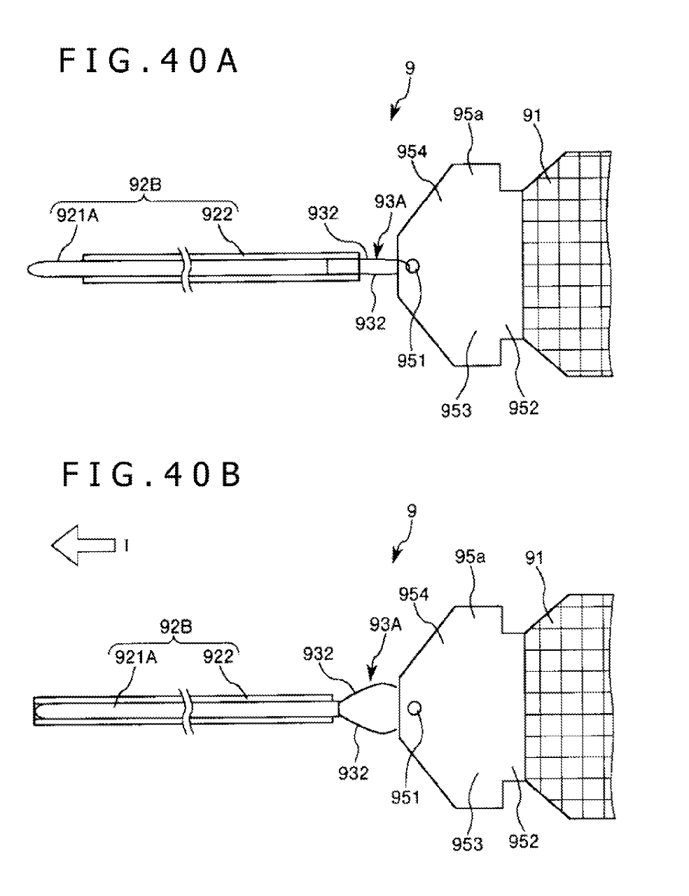

FIGS. 40A and 40B are views for illustrating a procedure of placing indwelling an implant according to a tenth exemplary embodiment of the present disclosure. Referring to these figures, the tenth exemplary embodiment of the implant and a method of placing an implant indwelling according to the present disclosure will be described below. The following description will center on differences from the above-described exemplary embodiments, and descriptions of the same items as those mentioned above will be omitted.

This exemplary embodiment is the same as the ninth exemplary embodiment above, except for a difference in the configuration (shape) of the interlock portion.

As shown in FIG. 40A, a linear body 921A of a guide portion 92B has a configuration wherein the aforementioned large outside diameter portion 924 is omitted.

In addition, an interlock portion 93A is composed essentially of two elastic wires 932 fixed to a proximal portion of the linear body 921A. The elastic wires 932 are curved so that their proximal portions come closer to each other. In addition, the elastic wires 932 have their proximal end portions spaced from each other in a natural state.

The linear body 921A provided with the elastic wires 932 as above is inserted into a tubular body 922, whereby the elastic wires 932 are restricted by a tube wall of the tubular body 922, and are deformed in directions for coming closer to each other. Consequently, the proximal portions of the elastic wires 932 make contact with each other. In this case, one of the elastic wires 932 is preliminarily passed through a through-hole 951 formed in an anchor portion 95a, whereby the guide portion 92B and an implant main body 91 are interlocked to each other.

In addition, when the tubular body 922 is moved in a direction of arrow I in FIG. 40B, the restriction on the elastic wires 932 by the tubular body 922 is released. As a result, the interlocking between the guide portion 92B and the implant main body 91 is released.

While the implant and the method of placing the implant indwelling according to the present disclosure have been described above referring to the exemplary embodiments illustrated in the drawings, the disclosure is not limited to the above exemplary embodiments. Each of the components can be replaced by a component having such a configuration as to have an equivalent function. In addition, an arbitrary structure or structures may be added to the original structures according to the present disclosure.

In addition, while the needle body is detachably held on the sheath main body in the above exemplary embodiments, this configuration is not limited. For example, a configuration in which the needle body is fixed to the sheath main body, such as a configuration wherein the sheath main body and the needle body are integrally formed, may also be adopted. In that case, after a living body is punctured by the puncture member and the needle body is made to protrude to the outside of the living body, the needle body may be cut by use of a pair of scissors or the like, whereby the distal end opening of the sheath main body can be opened.

While a configuration in which the sheath main body can be separated into a distal split piece and a proximal split piece has been described in the above exemplary embodiments, this configuration of the sheath main body is not limited. A configuration in which a distal end portion and a proximal end portion of the sheath main body are not separable from each other may also be adopted. For example, the sheath main body maybe configured to be a single tube. In that case, the aforementioned state-maintaining mechanism is also omitted.

While the sheath is configured as part of the puncture member in the above exemplary embodiments, this is not limited. For example, the sheath may be used in the manner of being inserted into a through-hole, which has preliminarily been formed in a living body, by use of some means. This will be described for example below, correspondingly to the aforementioned first exemplary embodiment. A puncture apparatus 1 wherein the aforementioned puncture member 3 is omitted is prepared. Using the insertion portion 71 as a puncture member, the distal portion 711 of the insertion portion 71 is made to puncture the patient's right-hand inguinal region. The distal portion 711 is then made to pass sequentially the obturator foramen on one side, a region between the urethra and the vagina, and the obturator foramen on the other side, and is thereafter protruded to the outside of the body via the left-hand inguinal region. Next, the insertion portion 71 is inserted into the inside, the sheath 30 (sheath main body 31) is advanced into the body along the insertion portion 71, to put the sheath 30 (sheath main body 31) into a state of having both its ends protruding from the body surface H. Next, the insertion portion 71 is pulled out of the body, thereby the sheath 30 is disposed in the living body. Then, the implant main body is disposed in the sheath 30, and the sheath 30 is pulled out of the body. By these operations, the implant main body can be placed indwelling in a living body, like in the aforementioned exemplary embodiment.

In addition, a procedure as follows may also be adopted. For example, the distal portion 711 of the insertion portion 71 is made to puncture the patient's right-hand inguinal region. The distal portion 711 is then made to pass sequentially the obturator foramen on one side, a region between the urethra and the vagina, and the obturator foramen on the other side, and is protruded to the outside of the body via the left-hand inguinal region. Thereafter, a distal portion of the sheath 30 is fixed to the distal portion 711. Next, the distal portion 711 is rotated in the reverse direction, whereby the insertion portion 71 is pulled out of the body, with the sheath 30 left indwelling in the living body. Then, the implant main body 91 is disposed in the sheath 30, and the sheath 30 is pulled out of the body. By these operations, the implant main body can be set indwelling in a living body, like in the aforementioned embodiments.

While a configuration in which the main body of the puncture member is disposed in a living body and thereafter the implant 9 is inserted into the main body has been described in the aforementioned exemplary embodiments, this configuration is not limited. The implant 9 may be accommodated in the puncture member (main body) from the beginning. In that case, it is preferable, for example, that an interlock portion located on the needle tip side is preliminarily fixed to the needle tip, which can help ensure that when the needle tip is detached from the main body, the interlock portion can be assuredly protruded to the outside of the main body, attendantly on the detachment. Consequently, the subsequent fine control of the layout of the implant main body 91 and the like can be carried out smoothly.

In addition, while the case where the puncture apparatus is applied to an implant for treatment of female urinary incontinence has been described in the above exemplary embodiments, this is not restrictive of the use of the implant.

Examples of which the present disclosure is applicable can include pelvic floor diseases inclusive of excretory disorders (urinary urgency, frequent urination, urinary incontinence, fecal incontinence, urinary retention, dysuria, etc.), pelvic organ prolapse, vesicovaginal fistula, urethrovaginal fistula, and pelvic pain, which would be attendant on weakening of the group of pelvic floor muscles. The pelvic organ prolapse include such diseases as cystocele, enterocele, rectocele, and hysterocele, or such diseases as anterior vaginal prolapse, posterior vaginal prolapse, vaginal apical prolapse, and vaginal vault prolapse, which are denominations based on classification of the vaginal wall part being prolapsed.

In addition, examples of overactive tissue can include the bladder, vagina, uterus, and bowels. Examples of lessactive tissue can include bones, muscles, fascias, and ligaments. For example, in relation to the pelvic floor diseases, examples of the lessactive tissue include obturator fascia, coccygeus fascia, cardinal ligament, uterosacral ligament, and sacrospinous ligament.

Examples of the procedure for interlocking an overactive tissue in the pelvic floor disorder with the lessactive tissue, can include a retropubic sling surgery, a transobturator sling surgery (Transobturator Sling Surgery, Transobturator Tape; TOT), a tension-free vaginal mesh (Tension-free Vaginal Mesh; TVM) surgery, a uterosacral ligament suspension (Uterosacral Ligament Suspension; USLS) surgery, an iliococcygeus fascia fixation surgery, and a coccygeus fascia fixation surgery.

The detailed description above describes an implant and a method of placing an implant indwelling. The disclosure is not limited, however, to the precise exemplary embodiments and variations described. Various changes, modifications and equivalents can effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. An implant to be placed indwelling in a living body, the implant inserted into a medical tube prior to being placed indwelling, the implant comprising:
an implant main body which is flexible, elongated, and shorter in length than the medical tube;
a guide portion which, at a time of insertion of the implant main body into the medical tube, is inserted into the medical tube prior to the implant main body and guides the implant main body; and
an interlock portion interlocking the implant main body and the guide portion to each other, the interlocking being released after the implant main body is inserted into the medical tube.

2. The implant according to claim 1,
wherein the interlock portion is loop-like in shape, and the interlocking between the implant main body and the guide portion is released by cutting a portion of the interlock portion.

3. The implant according to claim 1,
wherein at least part of the medical tube has a curved portion in a longitudinal direction, and
the guide portion is higher in rigidity than the implant main body, is deformable into a curved shape, and is deformed along a curved shape of the curved portion of the medical tube when guiding the implant main body.

4. The implant according to claim 1,
wherein the implant main body has an anchor portion which restricts movement in the longitudinal direction of the implant main body in a state in which the medical tube has been pulled out and the implant main body has been placed indwelling in the living body.

5. The implant according to claim 4,
wherein the anchor portion is flat plate-like in shape, and formed from a material higher in rigidity than a material constituting a network portion of the implant main body.

6. The implant according to claim 4,
wherein a maximum width of the anchor portion is smaller than a width of the network portion of the implant main body.

7. The implant according to claim 6,
wherein the anchor portion is divided into a small width portion interlocked to the network portion, a large width portion greater than the small width portion in width, and a decreasing-width portion, and wherein a width of the decreasing-width portion decreases along a direction towards a side opposite to the network portion.

8. The implant according to claim 7,
a step portion at a boundary portion between the small width portion and the large width portion.

9. The implant according to claim 4,
a through-hole in the anchor portion, which interlocks the guide portion to the implant main body.

10. The implant according to claim 4,
wherein the anchor portion has a distal portion of a decreasing-width portion, which is tapered into a sharp-pointed shape.

11. The implant according to claim 4,
wherein the anchor portion has rectangular cross-sectional shape and each corner of the anchor portion is rounded.

12. The implant according to claim 4,
wherein the anchor portion is ring-like in shape.

13. The implant according to claim 1, comprising:
a marker which indicates a position of the implant main body in the living body, the marker being provided on the interlock portion or the guide portion.

14. The implant according to claim 1,
wherein the medical tube is rigid to such an extent as to be able to maintain a lumen when left indwelling in the living body, and
the implant main body is more flexible than the medical tube.

15. The implant according to claim 1,
wherein a width of the implant main body decreases on both ends of the implant main body.

16. The implant according to claim 15,
wherein the implant main body has four corners, and each of the four corners has an angular portion.

17. The implant according to claim 1,
wherein the implant main body has a flat cross-section.

18. The implant according to claim 1,
wherein the implant main body has a circular cross-section.

19. A method of placing an implant indwelling in a living body by use of a medical tube in which the implant can be inserted, the method comprising:
placing the medical tube indwelling in the living body;
inserting the implant into the medical tube, and
thereafter pulling the medical tube out of the living body while leaving the implant in the living body so that the implant is placed indwelling in the living body.

20. The method according to claim 19,
wherein the implant includes:
an implant main body which is flexible, elongated, and shorter in length than the medical tube;
a guide portion which, at a time of insertion of the implant main body into the medical tube, is inserted into the medical tube prior to the implant main body and guides the implant main body; and
an interlock portion interlocking the implant main body and the guide portion to each other, the interlocking being released after the implant main body is inserted into the medical tube, the interlock portion being loop-like in shape; and
cutting a portion of the interlock portion to release the interlocking between the implant main body and the guide portion.
